SEQUENCE LISTING (34 sequences)

SEQ ID NO: 1 - RNA, 27 nt, synthetic construct
  misc_feature 1..27: note = nucleic acid
  misc_feature 5: note = r is a or g
  misc_feature 7: note = r is a, c, or u
  misc_feature 14: note = r is c or u
  misc_feature 27: note = r is a or g
  source 1..27: mol_type = other RNA
  misc_feature 8: note = r is a, c or u
ccagrtrrgagccrgggagctctctgr

SEQ ID NO: 2 - RNA, 27 nt, synthetic construct
  misc_feature 1..27: note = nucleic acid
  source 1..27: mol_type = other RNA
ccagatctgagcctgggagctctctgg

SEQ ID NO: 3 - RNA, 50 nt, synthetic construct
  misc_feature 1..50: note = nucleic acid
  source 1..50: mol_type = other RNA
ggtctctctggttagaccagatctgagcctgggagctctctggctaacta

SEQ ID NO: 4 - RNA, 51 nt, synthetic construct
  misc_feature 1..51: note = nucleic acid
  source 1..51: mol_type = other RNA
gtctctctggttagaccagatctgagcctgggagctctctggctaactagg

SEQ ID NO: 5 - RNA, 51 nt, synthetic construct
  misc_feature 1..51: note = nucleic acid
  source 1..51: mol_type = other RNA
tctggttagaccagatctgagcctgggagctctctggctaactagggaacc

SEQ ID NO: 6 - RNA, 50 nt, synthetic construct
  misc_feature 1..50: note = nucleic acid
  source 1..50: mol_type = other RNA
agcttgccttgagtgcttcaagtagtgtgtgcccgtctgttgtgtgactc

SEQ ID NO: 7 - RNA, 51 nt, synthetic construct
  misc_feature 1..51: note = nucleic acid
  source 1..51: mol_type = other RNA
agtagtgtgtgcccgtctgttgtgtgactctggtaactagagatccctcag

SEQ ID NO: 8 - RNA, 51 nt, synthetic construct
  misc_feature 1..51: note = nucleic acid
  source 1..51: mol_type = other RNA
aaatctctagcagtgggcccgaacagggacctgaaagcgaaagggaaacca

SEQ ID NO: 9 - RNA, 51 nt, synthetic construct
  misc_feature 1..51: note = nucleic acid
  source 1..51: mol_type = other RNA
atctctagcagtggcgcccgaacagggacctgaaagcgaaagggaaaccag

SEQ ID NO: 10 - RNA, 51 nt, synthetic construct
  misc_feature 1..51: note = nucleic acid
  source 1..51: mol_type = other RNA
aaaccagaggagctctctcgacgcaggactcggcttgctgagcgcgcacgg

SEQ ID NO: 11 - RNA, 51 nt, synthetic construct
  misc_feature 1..51: note = nucleic acid
  source 1..51: mol_type = other RNA
gaggagctctctcgacgcaggactcggcttgctgaagcgcgcacggcaaga

SEQ ID NO: 12 - RNA, 51 nt, synthetic construct
  misc_feature 1..51: note = nucleic acid
  source 1..51: mol_type = other RNA
gaggagctctctcgacgcaggactcggcttgctgagcgcgcacggcaagag

SEQ ID NO: 13 - RNA, 51 nt, synthetic construct
  misc_feature 1..51: note = nucleic acid
  source 1..51: mol_type = other RNA
ggcttgctgagcgcgcacggcaagaggcgaggggcggcgactggtgagtac

SEQ ID NO: 14 - RNA, 51 nt, synthetic construct
  misc_feature 1..51: note = nucleic acid
  source 1..51: mol_type = other RNA
gagcgcgcacggcaagaggcgaggggcggcgactggtgagtacgccaaaaa

SEQ ID NO: 15 - RNA, 51 nt, synthetic construct
  misc_feature 1..51: note = nucleic acid
  source 1..51: mol_type = other RNA
cggcaagaggcgaggggcggcgactggtgagtacgccaaaaattttgacta

SEQ ID NO: 16 - RNA, 51 nt, synthetic construct
  misc_feature 1..51: note = nucleic acid
  source 1..51: mol_type = other RNA
tgactagcggaggctagaaggagagagatgggtgcgagagcgtcagtatta

SEQ ID NO: 17 - RNA, 51 nt, synthetic construct
  misc_feature 1..51: note = nucleic acid
  source 1..51: mol_type = other RNA
agcggaggctagaaggagagagatgggtgcgagagcgtcagtattaagcgg

SEQ ID NO: 18 - RNA, 51 nt, synthetic construct
  misc_feature 1..51: note = nucleic acid
  source 1..51: mol_type = other RNA
gctagaaggagagagatgggtgcgagagcgtcagtattaagcgggggagaa

SEQ ID NO: 19 - RNA, 2574 nt, synthetic construct
  misc_feature 1..2574: note = nucleic acid
  source 1..2574: mol_type = other RNA
cctggaaagtccccagcggaaagtcccttgtagcaagctcgatgtcagcagttcttgaagtactccggatgcagctctcgggccacgtgatgaaatgctaggcggctgtcaaacctccactctaacacttctctctcagggtcatccattccatgcaggctcacagggtgtaacaagctggtgttctctcctttattggcctcttctaccttatctggctcaactggtactagcttgtagcaccatccaaaggtcagtggatatctgacccctggccctggtgtgtagttctgccaatcagggaagtagccttgtgtgtggtagatccacagatcaaggatatcttgtcttctttgggagtgaattagcccttccagtccccccttttcttttaaaaagtggctaagatctacagctgccttgtaagtcattggtcttaaaggtacctgaggtgtgactggaaaacccacctcttcctcctcttgtgcttctagccaggcacaagcagcattgttagctgctgtattgctacttgtgattgctccatgtttttctaggtctcgagatactgctcccaccccatctgctgctggctcagctcgtctcattctttcccttacagcaggccatccaatcacactactttttgaccacttgccacccatcttatagcaaaatcctttccaagccctgtcttattcttctaggtatgtggcgaatagctctataagctgcttgtaatacttctataaccctatctgtcccctcagctactgctatggctgtggcattgagcaagttaacagcactattctttagttcctgactccaatactgtaggagattccaccaatatttgagggcttcccaccccctgcgtcccagaagttccacaatcctcgttacaatcaagagtaagtctctcaagcggtggtagctgaagaggcacaggctccgcagatcgtcccagataagtgctaaggatccgttcactaatcgaatggatctgtctctgtctctctctccaccttcttcttctattccttcgggcctgtcgggtcccctcgggattgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcatgttattccaaatctgttccagagatttattactccaactagcattccaaggcacagcagtggtgcaaatgagttttccagagcaaccccaaatccccaggagctgttgatcctttaggtatctttccacagccaggattcttgcctggagctgtttgatgccccagactgtgagttgcaacagatgctgttgcgcctcaatagccctcagcaaattgttctgctgctgcactatatcagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatcgcctcctccaggtctgaagatctcggacccattgttgttattaccaccatctcttgttaatagcagcccagtaatatttgatgaacatctaatttgtccactgatgggaggggcatacattgcttttcctacttcctgccacatgtttataaattgttttattctgcatgggagtgtgattgtgtcacttccttcagtgttatttgacccttcagtactccaagtactattaaaccaagtactattaaacagttgtgttgaattacagtagaaaaattcccctccacaattaaaactgtgcgttacaatttctgggtcccctcctgaggattgcttaaagattattgttttattatttccaaattgttctcttaatttgctagctatctgttttaaagtggcattccattttgctctactaatgttacaatgtgcttgtctcatatttcctatttttcctattgtaacaaatgctctccctggtcccctctggatacggatactttttcttgtattgttgttgggtcttgtacaattaatttctacagatgtgttcagctgtactattatggttttagcattgtctgtgaaattggcagatctaattactacatcttcttctgctagactgccatttaacagcagttgagttgatactactggcctgattccatgtgtacattgtactgtgctgacatttgtacatggtcctgttccattgaacgtcttattattacattttagaatcgcaaaacc

SEQ ID NO: 20 - RNA, 570 nt, synthetic construct
  misc_feature 1..570: note = nucleic acid
  source 1..570: mol_type = other RNA
atgccccagactgtgagttgcaacagatgctgttgcgcctcaatagccctcagcaaattgttctgctgctgcactatatcagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatcgcctcctccaggtctgaagatctcggacccattgttgttattaccaccatctcttgttaatagcagcccagtaatatttgatgaacatctaatttgtccactgatgggaggggcatacattgcttttcctacttcctgccacatgtttataaattgttttattctgcatgggagtgtgattgtgtcacttccttcagtgttatttgacccttcagtactccaagtactattaaaccaagtactattaaacagttgtgttgaattacagtag

SEQ ID NO: 21 - RNA, 2271 nt, synthetic construct
  misc_feature 1..2271: note = nucleic acid
  source 1..2271: mol_type = other RNA
agttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcagggctgtttcgatgcttgcctgtgtcatgcccggaggacctgctggtggaggaattggtggacgggctattatccttggaggaagagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaataattgcgtgcttggtttacagggatgactcagggtttataagagagtaatgggggtatctgacgcgcgaggggaggtgtcgtagctgacggaggatgcatggtcctgcaaggataacaagaaggagtagcgcgacaagggtgattccagtttgtaaggcctctcgagcccactgtgagaggccaaggtcccagttaaggccccagccagtcaggactcgattctcaagggggggtctttcttgtagtattgagacatgggaattagtaatattcagaaaacagcactgttcttgtaatgctttgcataatcctccttgctcccagaacaggagatcaaggcctcgtctgttctgggcagcatactgcgcaattttgagtagatttttgtggtttttgactattgcttgagttaactgggaaatatctttgtccacctcatgtaggaggctctttcctgaggcgagggacatggagccggtaatcccgccagccactccggctcccatggccagggcggagacaagccagaccgccaccggtaccgctcggcgggagcgggatcctagggtgggaacaggtgacaaggaaaaggggggcaggatgagggagttatgacagggggaggagactatagcttgaatctgggggtcaaagcagtgggtccagttaaatggtaacgtcaggtggggggctggaagcgctaacgatgggtaaaggaggggggtagaagaagaggatggaacagagacgttgggagagtataggacgtgccaagtggagaggctggcacgatcgatacagacaatgcaagtataattagtgctttgtagggttaactggacaagggtcaggagttttgatttccatggtatagagggctcgaggatgtggtctaggttagagtgggggagtagaggaggggcggtgggaggcagttggctgggttcggtattaaggaaccagatggggtcatatcctggagcgtcgactagaagggagaagggaaaaccgcattttgaaaaatggagattaatattgaggcgtgaaacttcttgagtaaaattgacatcgtgctgaaacttccagtaggggctggagacggctcctgtataggggcaggtccatgattggcaccccaggtatgggcactttaaggaacaagggtctgaataagaggctgaataatagcctccgccatttcggtttggctttttaatccaatgagggaatagatatagggaataggtggcatggtagctggagtaacttactaggttagggcaggggggctgtagggcctgatctgctgaaagggccagcaggtcgagggtccacgaacaaactggctgggcaggattgcagggtttagagtggtatgaggagactccaattgtgagagtacagcagctggggctgtaatcaccgaagatgagggggcagaactggaagaataaaatcaaagtggcgagaaactt

SEQ ID NO: 22 - RNA, 621 nt, synthetic construct
  misc_feature 1..621: note = nucleic acid
  source 1..621: mol_type = other RNA
atggcggcctcagggctgtttcgatgcttgcctgtgtcatgcccggaggacctgctggtggaggaattggtggacgggctattatccttggaggaagagttaaaggacaaggaggaggagaaagctgtgcttgacggtttgctatccttagaagaggaaagccgcggccggctgcgacggggccctccaggggagaaagcgccacctcgcggggaaacgcatcgtgatcggcagcgacgggctgaggagaagaggaagcgaaaaaaagagcgggagaaagaggaggaaaagcagattgctgagtatttgaaaaggaaggaagaggagaaggcacggcgcaggaggcgggcggagaagaaggccgctgacgtcgccaggaggaagcaggaagagcaggagcgccgtgagcgcaagtggagacaaggggctgagaaggcgaaacagcatagtgctaggaaagaaaaaatgcaggagttggggattgatggctatactagacagttggaaggcgaggtggagtccttggaggctgaacggaggaagttgctgcaggagaaggaggatttgatgggagaggttaattattggcaggggaggctggaggcgatgtggttgcaataa

SEQ ID NO: 23 - DNA, 21 bp, synthetic construct
  misc_feature 1..21: note = nucleic acid
  source 1..21: mol_type = other DNA
caacagacgggcacacactac

SEQ ID NO: 24 - DNA, 21 bp, synthetic construct
  misc_feature 1..21: note = nucleic acid
  source 1..21: mol_type = other DNA
caacagacgggcacacactac

SEQ ID NO: 25 - DNA, 24 bp, synthetic construct
  misc_feature 1..24: note = nucleic acid
  source 1..24: mol_type = other DNA
ccactgctagagattttccacact

SEQ ID NO: 26 - DNA, 23 bp, synthetic construct
  misc_feature 1..23: note = nucleic acid
  source 1..23: mol_type = other DNA
ccccctggccttaaccgaatttt

SEQ ID NO: 27 - DNA, 25 bp, synthetic construct
  misc_feature 1..25: note = nucleic acid
  source 1..25: mol_type = other DNA
tatctctatcctttgatgcacacaa

SEQ ID NO: 28 - DNA, 18 bp, synthetic construct
  misc_feature 1..18: note = nucleic acid
  source 1..18: mol_type = other DNA
gggcgccactgctagaga

SEQ ID NO: 29 - DNA, 29 bp, synthetic construct
  misc_feature 1..29: note = nucleic acid
  source 1..29: mol_type = other DNA
caaatttctactaatgcttttattttttc

SEQ ID NO: 30 - DNA, 20 bp, synthetic construct
  misc_feature 1..20: note = nucleic acid
  source 1..20: mol_type = other DNA
tgggataagggtctgaaacg

SEQ ID NO: 31 - DNA, 21 bp, synthetic construct
  misc_feature 1..21: note = nucleic acid
  source 1..21: mol_type = other DNA
gaagcaggcaaaggaccgagg

SEQ ID NO: 32 - DNA, 17 bp, synthetic construct
  misc_feature 1..17: note = nucleic acid
  source 1..17: mol_type = other DNA
ggcccggagaccagacg

SEQ ID NO: 33 - DNA, 20 bp, synthetic construct
  misc_feature 1..20: note = nucleic acid
  source 1..20: mol_type = other DNA
caacagacgggcacacacac

SEQ ID NO: 34 - DNA, 21 bp, synthetic construct
  misc_feature 1..21: note = nucleic acid
  source 1..21: mol_type = other DNA
agcccaaaaagcgccgaggcc

(19) United States
(12) Patent Application Publication
(10) Pub. No.: US 2025/0034664 A1
(43) Pub. Date: Jan. 30, 2025

(54) Compositions and Methods for Diagnosis and Therapy of Viral Infection

(71) Applicant: George Mason Research Foundation, Inc., Fairfax, VA (US)

(72) Inventors: Fatah Kashanchi, Potomac, MD (US); Lance Liotta, Bethesda, MD (US); Pooja Khatkar, Mclean, VA (US)

(21) Appl. No.: 18/705,295

(22) PCT Filed: Oct. 26, 2022
(86) PCT No.: PCT/US2022/078711

§ 371 Date: Apr. 26, 2024

Related U.S. Application Data
(60) Provisional application No. 63/272,360, filed on Oct. 27, 2021.

(51) Int. Cl. C12Q 1/70; C12Q 1/6851

(52) U.S. Cl. CPC C12Q 1/703; C12Q 1/6851

(57) ABSTRACT

Compositions that comprise extracellular portions of viral nucleic acids present on plasma membranes of host cells infected with virus, or that comprise molecules that specifically bind the extracellular portions, are disclosed, and provide for targeting cells that harbor the virus, in particular cells that harbor latent virus. Reagents, components, and kits as well as diagnostic and therapeutic methods based on the extracellular portions of viral nucleic acids that identify cells harboring the virus, in particular harboring latent virus such as, for example, latent retrovirus (e.g., HIV), are also disclosed.

### CROSS-REFERENCE TO RELATED APPLICATIONS

This application is a U.S. National Phase application and claims the benefit of priority under 35 U.S.C. § 371 of International Application No. PCT/US2022/078711, filed Oct. 26, 2022, which claims priority to U.S. Provisional Application No. 63/272,360 filed Oct. 27, 2021. The entire contents of each of these applications are incorporated herein by reference.

### STATEMENT REGARDING FEDERALLY SPONSORED RESEARCH OR DEVELOPMENT

This invention was made with government support under grant number NS099029 awarded by the National Institutes of Health. The government has certain rights in the invention.

### REFERENCE TO AN ELECTRONIC SEQUENCE LISTING

The Sequence Listing submitted herewith as a xml file named “381789-7004US1” created on Apr. 26, 2024 and having a size of 52, 196 bytes, is incorporated herein by reference in its entirety.

### SUMMARY OF THE INVENTION

In one aspect, the present invention provides a composition comprising an extracellular portion, or a molecule that specifically binds the extracellular portion, of a viral nucleic acid present on a plasma membrane of a host cell infected with a virus.

In another aspect, the present invention provides a method for treating or preventing infection by a virus. The method comprises administering a therapeutically or prophylactically effective amount of a composition to a subject, wherein the composition comprises an extracellular portion, or a molecule that specifically binds the extracellular portion, of a viral nucleic acid present on a plasma membrane of a host cell infected with a virus, thereby treating or preventing infection by the virus in the subject.

In some aspects, the present invention provides a method for identifying a subject infected with a virus. The method comprises detecting the presence of an extracellular portion of a viral nucleic acid present on a plasma membrane of a host cell infected with a virus using a composition, wherein the composition comprises an extracellular portion, or a molecule that specifically binds the extracellular portion, of a viral nucleic acid present on a plasma membrane of a host cell infected with a virus, thereby identifying the subject infected with the virus.

In other aspects, the present invention provides a method for separating or killing virally infected cells ex vivo. The method comprises contacting cells obtained from a subject with a composition comprising a molecule that specifically binds the extracellular portion of a viral nucleic acid present on a plasma membrane of a host cell infected with a virus, wherein the virally infected cells bind to a molecule.

In one aspect, the present invention provides a molecule that specifically binds an extracellular portion of a viral nucleic acid present on a plasma membrane of a host cell infected with a virus.

In another aspect, the present invention provides an exosome, a liposome, a macrovesicle, or a nanoparticle that is coated with a molecule that specifically binds an extracellular portion of a viral nucleic acid present on a plasma membrane of a host cell infected with a virus.

### BRIEF DESCRIPTION OF THE DRAWINGS

The following detailed description of preferred embodiments of the invention will be better understood when read in conjunction with the appended drawings in one of the most studied Retrovirus called HIV-1. However, the invention is not limited to HIV-1 only, it is also applicable to, for example, Retroviruses transcribing RNA from 5′ or 3′ region of LTR. For the purpose of illustrating the invention, there are shown in the drawings embodiments which are presently preferred. It should be understood, however, that the invention is not limited to the precise arrangements and instrumentalities of the embodiments shown in the drawings.

The foregoing and other features and advantages of the present invention will be more fully understood from the following detailed description of illustrative embodiments taken in conjunction with the accompanying drawings.

FIG. 1 is a sequence alignment showing the sequence conservation of a trans-activation response element (TAR) of HIV-1. The sequences are depicted as DNA sequences, and potential sites of glycosylation on the 3 consecutive guanine nucleotides (vertical arrow) of the TAR sequence are underlined. Sequences are retrieved from Los Alamos HIV-1 Database.

FIG. 2 is an overview of sequence conservation in HIV-1 TAR (SEQ ID NO: 1). Highly and least conserved regions are shown by the double and single underlines, respectively.

FIGS. 3A-3D show RNA secondary structures of four HIV-1 TAR species ((A): TAR1 (GGG residues at position 31-33 are potential sugar binding residues); (B): TAR2; (C) TAR3; and (D) TAR4).

FIGS. 4A-4B are graphs showing different viral transcripts in different part of cells in HIV-1 infected (A) T-cells (J1.1) and (B) Myeloids (U1), with majority of them present in plasma membrane fractions. Nucleus (N)+Cytoplasm+Plasma membrane (PM)+EVs=100%. RNA from Nuclear, cytoplasmic, plasma membrane and extracellular vesicles fractions were isolated for myeloid cells (U1) and T-cells (J1.1), analyzed by RT-qPCR for the presence of TAR, Long LTR, Pol, env and nef transcripts using primers from Yukl et al. (see method section for primers used). Student's t-test was done to calculate the statistical significance w.r.t control ***, p<0.001. Error bars, S.D.

FIGS. 5A-5I are graphs of data from experiments and schematic showing presence of TAR in the membrane of infected cells. (A) Plasma membranes were isolated from ˜50 million cells of monocytes (U937), uninfected T-cells (Jurkat), HIV-1 infected monocytes (U1), and T-cells (J1.1) following the plasma membrane extraction kit protocol (abcam; 65400). RNA was isolated from plasma membrane pellets using Trizol Reagent, deglycosylated (sialidase enzymatic digestion) followed by RT-qPCR to quantify the TAR RNA copies in plasma membrane of myeloid cells and T-lymphocytes. Student's t-tests were used to calculate the statistical significance between uninfected and infected cells. (B) Myeloid cells (U937) and T-cells (CEM) were infected with HIV-1pNL4-3 DNA (20 ug) at 37° C. for 48 hours at followed by plasma membrane isolation, deglycosylation and RT-qPCR to confirm the translocation of TAR RNA in the plasma membrane of transfected cells. Student's t-test was used to calculate the statistical significance between uninfected and infected cells. *, p<0.05; **, p<0.01; ***, p<0.001. Error bars, S.D. (C): To test for the presence of Siglec (Glycans) molecules on TAR RNA surface, plasma membranes were isolated from infected Myeloids (U1) and T-cells (J1.1) and prepared for immunoprecipitation (IP). Each preparation was fractioned into three reactions. Five micrograms of each IgG and J2 (RNA binding antibody; Scicons) were used for IP in the first two reactions. In the third reaction, one ug of Human rec-hSiglec-14 was added to the samples (overnight) before immunoprecipitation with Siglec-5/Siglec-14 antibody (5 ug). Protein A/G beads were added the next day (2 hours, at 4° C.), and the IPed samples were washed. RNA was isolated for RT-qPCR using TAR primers. (E) and (I): Latent T_{CM} CD4+ cells (5 million) were purified using the Dynabeads CD4 Positive Isolation Kit (Thermo Fisher Scientific) and used for plasma membrane isolation, RNA isolation, and RT-qPCR using TAR primers. (D) and (H): To examine the presence of TAR RNA in the plasma membrane of latent macrophages, PBMCs from three different donors were treated with PHA/IL-2 and followed by infection with HIV-1 (89.6) and 3 days later treated with cART and IL-7 for one week. Suspension cells were removed and adherent cells (macrophages) were collected 24 hrs post PMA treatment (100 uM). Plasma membranes were isolated from PBMCs and subjected to total RNA isolation, followed by RT-qPCR using TAR primers. Student's t-tests was done to calculate the statistical significance between latent infected and uninfected T-cells. *, p<0.05; **, p<0.01; ***, p<0.001. (F) Top image: Plasma membrane and cytosolic fractions (25 ug) from uninfected Myeloid/T-cells (U937, Jurkat) and infected Myeloid/T-cells (U1, J1.1) were immunoblotted for the cytosolic marker, HSP-90 protein (NB100-1972) (Top image). *, p<0.05; **, p<0.01; ***, p<0.001. Error bars, S.D.; Bottom image: Uninfected CEM cells were treated with plasma membrane fractions derived from J1.1 or U1 cells (˜10⁹ copies of TAR RNA) and incubated for 72 hrs. The total cell lysate was then immunoblotted for viral antigen p24. U1 and CEM cell extracts served as controls (Bottom image). (G): Schematic representation of Glycosylated HIV-1 TAR RNA (non-coding RNA) on the cell surface of HIV-1 Infected Cell.

FIGS. 6A-6B are schematics illustrating HIV-1 infected cells displaying glycosylated viral non-coding RNA (TAR RNA) on surfaces. The RNA type could be any form presented in FIG. 3A-3D.

FIGS. 7A-7D show increased surface expression of dsRNA on cells as shown using flow cytometry. CEM and Jurkat cells were used to determine expression levels of dsRNA on human T-cells following HIV infection using Anti-dsRNA monoclonal antibody J2. Dot plots show fluorescent intensities of the gated population of cells that were left untreated (CEM cells: A, Jurkat cells: C) as well as cells that were infected with HIV (CEM cells: B, Jurkat cells: D). Histograms are showing a shift in fluorescence in the FITC channel (Q4) between the untreated and HIV-infected cells. End panels towards the right are representative of control cells showing background fluorescence as they were only stained with the secondary Donkey anti-mouse Alexa-488 antibody. Samples were stained using a primary antibody against dsRNA (anti-dsRNA J2) followed by a donkey anti-mouse Alexa-488 secondary antibody and analyzed using a BD FACSaria II Flow Cytometer.

FIGS. 8A-8C show presence of various TAR non-coding RNAs in plasma membrane of HIV-1 infected cells. (A) The schematic representation of length and location of four transcripts on HIV-1 genome determined by RNA sequencing. Plasma membrane fractions were isolated from (B) myeloid cells (U1) and (C) T-cells (J1.1) and examined for the presence of four non-coding transcripts by RT-qPCR using reverse primers for TAR 1, TAR2, TAR 3, and TAR 4 (see method section for primer used). The secondary structures of four non-coding RNAs was predicted from RNAfold server are shown in FIGS. 3A-3D.

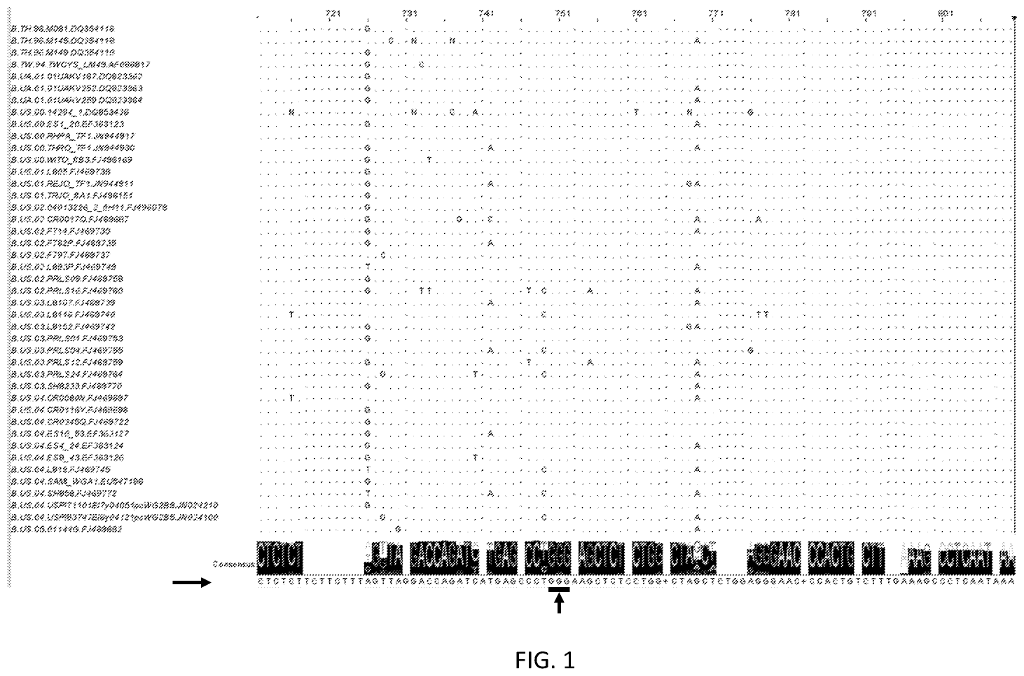

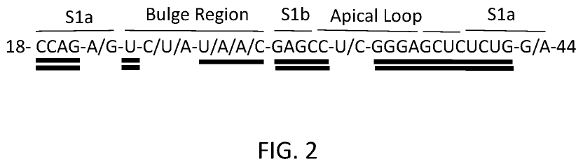

### DETAILED DESCRIPTION OF THE INVENTION

Although the methods described herein may be disclosed and described as step(s), it is to be understood that the methods are not necessarily limited by the order of steps, as some steps may, in accordance with these methods, occur in different orders, and/or concurrently with other step(s) described herein and/or known in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to one of ordinary skill in the art, and are explained fully in the literature. Sec, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook, and Harlow et al., Antibodies: A Laboratory Manual, Chapter 14, Cold Spring Harbor Laboratory, Cold Spring Harbor (2013, 2nd edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of the term “including,” as well as other forms, such as “includes” and “included,” is not limiting. The transitional phrase “consisting of” does not include elements, steps or ingredients not expressly recited in the claim. The transitional phrase “consisting essentially of” limits the scope of the claims to those that do not significantly affect the material or steps specified and the fundamental and novel features of the claimed invention. A “consisting essentially of” claim is located between a closed claim expressed in the “consisting of” format and a fully open claim written in the “comprising” format.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, linear antibodies, and multi-specific antibodies formed from antibody fragments as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody, where the whole molecule is of human origin or consists of an amino acid sequence identical to a human form of the antibody.

As used herein, the term “antigen” refers to any substance capable of eliciting an immune response. This immune response may involve either antibody production, or the activation of specific immunologically-competent cells, or both. The skilled artisan will understand that any substance, including nucleic acids (e.g., viral nucleic acids (e.g., viral RNA), or a portion thereof (e.g., extracellular portion)), can serve as an antigen.

As used herein, the term “epitope” refers to the parts of an antigen that contact the antigen binding site of an antibody and/or a receptor of an immunologically-competent cell (e.g., a T cell receptor).

As used herein, the term “specifically binds,” refers to the interaction between binding pairs (e.g., an antibody and an antigen).

“Variant” as used herein when referring to a sequence (e.g., nucleic acid or amino acid sequence) is used to refer to a sequence comprising an alteration or modification, including e.g., but not limited to, a substitution, insertion, deletion, addition, fusion, and/or glycosylation, at one or more residue positions, as compared to a reference sequence. Examples of reference sequences include naturally occurring unaltered or unmodified polynucleotides and polypeptides, e.g., naturally occurring polynucleotides and polypeptides, from viral species. In certain embodiments, the reference polynucleotide or polypeptide, is a naturally occurring polynucleotide or polypeptide having the closest sequence identity or homology with the variant polynucleotide or polypeptide to which it is being compared. In certain embodiments, the reference polynucleotide or polypeptide is a parental molecule having a naturally occurring or known sequence on which a mutation has occurred or has been made to arrive at the variant polynucleotide or polypeptide.

The term “viral nucleic acid” refers to the genome of a virus, or a portion thereof (or, in the case of viruses whose genome comprises multiple segments, any of the segments or a portion of such a segment). The term encompasses both RNA and DNA forms of such nucleic acids and molecules having complementary sequences. DNA molecules identical to or complementary to viral RNA nucleic acids are considered viral nucleic acids, and RNA molecules identical to or complementary to viral DNA nucleic acids are considered viral nucleic acids, it being understood that DNA and RNA will contain T and U, respectively, at corresponding positions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In one aspect, the present invention provides a composition comprising an extracellular portion, or a molecule that specifically binds the extracellular portion, of a viral nucleic acid present on a plasma membrane of a host cell infected with a virus.

In some embodiments, the host cell is a human cell. In other embodiments, the host cell is a nonhuman cell of a mammal (e.g. dog, cat pig, cow, horse, goat, rabbit, rodent), a bird (e.g. chicken, turkey, duck, goose), or a fish.

Some viruses have two stages, namely a lytic stage when the virus can be infectious and a latent life cycle when the virus can persist in the host. For example, during retrovirus infection, viral genome becomes quiescent or latent in the nucleus, a state in which virus is not replicating. At any time, cells containing the latent reservoir can become active again and start making more viral copies. For instance, latent HIV-1 (retrovirus) reservoirs are established early during primary infection in CD4+ T cells.

In one embodiment, the viral nucleic acid is expressed in a latent life cycle of the virus. Many DNA and RNA viruses synthesize their own noncoding RNA (ncRNA). In some cases, diverse biological roles, including the regulation of viral replication, viral persistence, host immune evasion, and cellular transformation, have been attributed to viral ncRNAs.

In some embodiments, the viral nucleic acid comprises a ncRNA.

In another embodiment, the viral nucleic acid comprises a ncRNA transcribed from a 5′ LTR or a 3′ LTR end of a virus. In one embodiment, the viral nucleic acid comprises a ncRNA transcribed from the 5′ LTR of HIV. In some embodiments, the viral nucleic acid comprises a ncRNA transcribed from the 3′ LTR of HIV. In other embodiments, the viral nucleic acid comprises a ncRNA transcribed from the 5′ LTR of HTLV. In some embodiment, the viral nucleic acid comprises a ncRNA transcribed from the 3′ LTR of HTLV.

In other embodiments, the viral nucleic acid is glycosylated. In some embodiments, at least the extracellular portion of the viral nucleic acid is glycosylated.

In one embodiment, the viral nucleic acid comprises a N-glycan. In another embodiment, the viral nucleic acid comprise an O-glycan. In other embodiments, the viral nucleic acid comprises a sialylated glycan.

In another embodiment, the glycosylation comprises one or more of glucose, galactose, mannose, fucose, N-acetylgalactosamine (GalNAc), N-acetylglucosamine (GlcNAc) and sialic acid (e.g., N-acetyl-neuraminic acid (NANA)).

In some embodiments, the viral nucleic acid comprises N-glycans containing sialic acid (including NANA, N-glycolylneuraminic acid (NGNA) and their analogues and derivatives). In one embodiment, the viral nucleic acid comprises a mixture of α-2,3- and α-2,6-linked sialic acids. In another embodiment, the viral nucleic acid comprises only α-2,6-linked sialic acid. In one embodiment, the viral nucleic acid comprises α-2,6-linked sialic acid and does not contain a detectable amount of α-2,3-linked sialic acid. In one embodiment, sialic acid is NANA or N-NGNA, or a mixture thereof. In another embodiment, sialic acid is an analog or derivative of NANA or NGNA with acetylation at position 9 on sialic acid. In one embodiment, the N-glycans on the viral nucleic acid comprises NANA and do not contain NGNA.

In one embodiment, the N-glycans on the viral nucleic acid comprises a mixture of fucosylated and non-fucosylated N-glycans. In another embodiment, the N-glycans on the viral nucleic acid do not contain fucose.

There are a number of different classes of viruses known in the art that can infect the host cell. For example, class I and class II DNA viruses contain nucleic acid in a double-stranded and single-stranded genome, respectively. Classes III (reovirus), IV (poliovirus), V (vesicular stomatitis virus), and VI (retroviruses) are RNA viruses.

In some embodiments, the virus is a retrovirus.

Retroviruses are enveloped viruses that belong to the viral family Retroviridae. Retroviruses include the genus of lentivirus (e.g., human immunodeficiency viruses (HIV-1 and HIV-2), simian immunodeficiency virus (SIV), feline immunodeficiency virus (FIV), equine infectious anaemia virus (EIAV)), the genus of alpha retrovirus (e.g., avian leukosis virus (ALV)), the genus of beta retrovirus (e.g., mouse mammary tumor virus (MMTV)), the genus of gamma retrovirus (e.g., murine leukemia virus (MLV)), the genus of delta retrovirus (e.g., bovine leukemia virus (BLV), human T-lymphotropic virus (HTLV)), the genus of Epsilon retrovirus (e.g., Walleye dermal sarcoma virus), and the genus of Lentivirus.

In some embodiments, the virus is a lentivirus. In one embodiment, the virus is HIV. In other embodiments, the virus is HIV-1 or HIV-2. In one embodiment, the virus is HIV-1. In another embodiment, the virus is HIV-2.

In one embodiment, the viral nucleic acid is a ncRNA present on the plasma membrane of a human cell infected with HIV-1, wherein the ncRNA is an HIV-1 ncRNA expressed in a latent life cycle of the HIV-1.

In some embodiments, the viral nucleic acid comprises a TAR sequence, or a variant sequence thereof. In one embodiment, the TAR is a HIV-1 TAR.

In another embodiment, the viral nucleic acid comprises the nucleotide sequence as set forth in:

(SEQ ID NO: 1)
CCAGX₁UX₂X₃GAGCCX₄GGGAGCUCUCUGX₅

1), wherein X₁ is A or G; X₂ is C, U, or A; X₃ is U, A, or C; X₄ is U or C; and X₅ is G or A; or a variant sequence thereof;

(SEQ ID NO: 2)
CCAGAUCUGAGCCUGGGAGCUCUCUGG

or a variant sequence thereof;

(SEQ ID NO: 3)
GGUCUCUCUGGUUAGACCAGAUCUGAGCCUGGGAGCUCUCUGGCUAAC
UA

or a variant sequence thereof;

(SEQ ID NO: 4)
GUCUCUCUGGUUAGACCAGAUCUGAGCCUGGGAGCUCUCUGGCUAACUA
GG

or a variant sequence thereof;

(SEQ ID NO: 5)
UCUGGUUAGACCAGAUCUGAGCCUGGGAGCUCUCUGGCUAACUAGGGAA
CC

or a variant sequence thereof;

(SEQ ID NO: 6)
AGCUUGCCUUGAGUGCUUCAAGUAGUGUGUGCCCGUCUGUUGUGUGACU
C

or a variant sequence thereof;

(SEQ ID NO: 7)
AGUAGUGUGUGCCCGUCUGUUGUGUGACUCUGGUAACUAGAGAUCCCU
CAG

or a variant sequence thereof;

(SEQ ID NO: 8)
AAAUCUCUAGCAGUGGGCCCGAACAGGGACCUGAAAGCGAAAGGGAAA
CCA

or a variant sequence thereof;

(SEQ ID NO: 9)
AUCUCUAGCAGUGGCGCCCGAACAGGGACCUGAAAGCGAAAGGGAAAC
CAG

or a variant sequence thereof;

(SEQ ID NO: 10)
AAACCAGAGGAGCUCUCUCGACGCAGGACUCGGCUUGCUGAGCGCGCA
CGG

or a variant sequence thereof;

(SEQ ID NO: 11)
GAGGAGCUCUCUCGACGCAGGACUCGGCUUGCUGAAGCGCGCACGGCA
AGA

or a variant sequence thereof;

(SEQ ID NO: 12)
GAGGAGCUCUCUCGACGCAGGACUCGGCUUGCUGAGCGCGCACGGCAA
GAG

or a variant sequence thereof;

(SEQ ID NO: 13)
GGCUUGCUGAGCGCGCACGGCAAGAGGCGAGGGGCGGCGACUGGUGAG
UAC

or a variant sequence thereof;

(SEQ ID NO: 14)
GAGCGCGCACGGCAAGAGGCGAGGGGCGGCGACUGGUGAGUACGCCAA
AAA

or a variant sequence thereof;

(SEQ ID NO: 15)
CGGCAAGAGGCGAGGGGCGGCGACUGGUGAGUACGCCAAAAAUUUUGA
CUA

or a variant sequence thereof;

(SEQ ID NO: 16)
UGACUAGCGGAGGCUAGAAGGAGAGAGAUGGGUGCGAGAGCGUCAGUA
UUA

or a variant sequence thereof;

(SEQ ID NO: 17)
AGCGGAGGCUAGAAGGAGAGAGAUGGGUGCGAGAGCGUCAGUAUUAAG
CGG

or a variant sequence thereof; or

(SEQ ID NO: 18)
GCUAGAAGGAGAGAGAUGGGUGCGAGAGCGUCAGUAUUAAGCGGGGGA
GAA

or a variant sequence thereof.

HIV-1 antisense protein (asp) transcript is disclosed by GenBank accession number JQ866626.1 (complete eds) and Kobayashi-Ishihara M. et al., “HIV-1-encoded antisense RNA suppresses viral replication for a prolonged period. Retrovirology,” 9:38 (2012), each of which is herein incorporated by reference in its entirety.

In one embodiment, the viral nucleic acid comprises HIV-1 antisense protein (asp) transcript, or a fragment or variant thereof.

In another embodiment, the viral nucleic acid comprises the nucleotide sequence as set forth in SEQ ID NO: 19:

CCUGGAAAGUCCCCAGCGGAAAGUCCCUUGUAGCAAGCUCGAUGUCAG
CAGUUCUUGAAGUACUCCGGAUGCAGCUCUCGGGCCACGUGAUGAAAU
GCUAGGCGGCUGUCAAACCUCCACUCUAACACUUCUCUCUCAGGGUCA
UCCAUUCCAUGCAGGCUCACAGGGUGUAACAAGCUGGUGUUCUCUCCU
UUAUUGGCCUCUUCUACCUUAUCUGGCUCAACUGGUACUAGCUUGUAG
CACCAUCCAAAGGUCAGUGGAUAUCUGACCCCUGGCCCUGGUGUGUAG
UUCUGCCAAUCAGGGAAGUAGCCUUGUGUGUGGUAGAUCCACAGAUCA
AGGAUAUCUUGUCUUCUUUGGGAGUGAAUUAGCCCUUCCAGUCCCCCC
UUUUCUUUUAAAAAGUGGCUAAGAUCUACAGCUGCCUUGUAAGUCAUU
GGUCUUAAAGGUACCUGAGGUGUGACUGGAAAACCCACCUCUUCCUCC
UCUUGUGCUUCUAGCCAGGCACAAGCAGCAUUGUUAGCUGCUGUAUUG
CUACUUGUGAUUGCUCCAUGUUUUUCUAGGUCUCGAGAUACUGCUCCC
ACCCCAUCUGCUGCUGGCUCAGCUCGUCUCAUUCUUUCCCUUACAGCA
GGCCAUCCAAUCACACUACUUUUUGACCACUUGCCACCCAUCUUAUAG
CAAAAUCCUUUCCAAGCCCUGUCUUAUUCUUCUAGGUAUGUGGCGAAU
AGCUCUAUAAGCUGCUUGUAAUACUUCUAUAACCCUAUCUGUCCCCUC
AGCUACUGCUAUGGCUGUGGCAUUGAGCAAGUUAACAGCACUAUUCUU
UAGUUCCUGACUCCAAUACUGUAGGAGAUUCCACCAAUAUUUGAGGGC
UUCCCACCCCCUGCGUCCCAGAAGUUCCACAAUCCUCGUUACAAUCAA
GAGUAAGUCUCUCAAGCGGUGGUAGCUGAAGAGGCACAGGCUCCGCAG
AUCGUCCCAGAUAAGUGCUAAGGAUCCGUUCACUAAUCGAAUGGAUCU
GUCUCUGUCUCUCUCUCCACCUUCUUCUUCUAUUCCUUCGGGCCUGUC
GGGUCCCCUCGGGAUUGGGAGGUGGGUCUGAAACGAUAAUGGUGAAUA
UCCCUGCCUAACUCUAUUCACUAUAGAAAGUACAGCAAAAACUAUUCU
UAAACCUACCAAGCCUCCUACUAUCAUUAUGAAUAAUUUUAUAUACCA
CAGCCAAUUUGUUAUGUUAAACCAAUUCCACAAACUUGCCCAUUUAUC
UAAUUCCAAUAAUUCUUGUUCAUUCUUUUCUUGCUGGUUUUGCGAUUC
UUCAAUUAAGGAGUGUAUUAAGCUUGUGUAAUUGUUAAUUUCUCUGUC
CCACUCCAUCCAGGUCAUGUUAUUCCAAAUCUGUUCCAGAGAUUUAUU
ACUCCAACUAGCAUUCCAAGGCACAGCAGUGGUGCAAAUGAGUUUUCC
AGAGCAACCCCAAAUCCCCAGGAGCUGUUGAUCCUUUAGGUAUCUUUC
CACAGCCAGGAUUCUUGCCUGGAGCUGUUUGAUGCCCCAGACUGUGAG
UUGCAACAGAUGCUGUUGCGCCUCAAUAGCCCUCAGCAAAUUGUUCUG
CUGCUGCACUAUAUCAGACAAUAAUUGUCUGGCCUGUACCGUCAGCGU
CAUUGACGCUGCGCCCAUAGUGCUUCCUGCUGCUCCCAAGAACCCAAG
GAACAAAGCUCCUAUUCCCACUGCUCUUUUUUCUCUCUGCACCACUCU
UCUCUUUGCCUUGGUGGGUGCUACUCCUAAUGGUUCAAUUUUUACUAC
UUUAUAUUUAUAUAAUUCACUUCUCCAAUUGUCCCUCAUAUCGCCUCC
UCCAGGUCUGAAGAUCUCGGACCCAUUGUUGUUAUUACCACCAUCUCU
UGUUAAUAGCAGCCCAGUAAUAUUUGAUGAACAUCUAAUUUGUCCACU
GAUGGGAGGGGCAUACAUUGCUUUUCCUACUUCCUGCCACAUGUUUAU
AAAUUGUUUUAUUCUGCAUGGGAGUGUGAUUGUGUCACUUCCUUCAGU
GUUAUUUGACCCUUCAGUACUCCAAGUACUAUUAAACCAAGUACUAUU
AAACAGUUGUGUUGAAUUACAGUAGAAAAAUUCCCCUCCACAAUUAAA
ACUGUGCGUUACAAUUUCUGGGUCCCCUCCUGAGGAUUGCUUAAAGAU
UAUUGUUUUAUUAUUUCCAAAUUGUUCUCUUAAUUUGCUAGCUAUCUG
UUUUAAAGUGGCAUUCCAUUUUGCUCUACUAAUGUUACAAUGUGCUUG
UCUCAUAUUUCCUAUUUUUCCUAUUGUAACAAAUGCUCUCCCUGGUCC
CCUCUGGAUACGGAUACUUUUUCUUGUAUUGUUGUUGGGUCUUGUACA
AUUAAUUUCUACAGAUGUGUUCAGCUGUACUAUUAUGGUUUUAGCAUU
GUCUGUGAAAUUGGCAGAUCUAAUUACUACAUCUUCUUCUGCUAGACU
GCCAUUUAACAGCAGUUGAGUUGAUACUACUGGCCUGAUUCCAUGUGU
ACAUUGUACUGUGCUGACAUUUGUACAUGGUCCUGUUCCAUUGAACGU
CUUAUUAUUACAUUUUAGAAUCGCAAAACC

or a variant or fragment thereof.

In another embodiment, the viral nucleic acid comprises the nucleotide sequence as set forth in SEQ ID NO: 20:

AUGCCCCAGACUGUGAGUUGCAACAGAUGCUGUUGCGCCUCAAUAGCC
CUCAGCAAAUUGUUCUGCUGCUGCACUAUAUCAGACAAUAAUUGUCUG
GCCUGUACCGUCAGCGUCAUUGACGCUGCGCCCAUAGUGCUUCCUGCU
GCUCCCAAGAACCCAAGGAACAAAGCUCCUAUUCCCACUGCUCUUUUU
UCUCUCUGCACCACUCUUCUCUUUGCCUUGGUGGGUGCUACUCCUAAU
GGUUCAAUUUUUACUACUUUAUAUUUAUAUAAUUCACUUCUCCAAUUG
UCCCUCAUAUCGCCUCCUCCAGGUCUGAAGAUCUCGGACCCAUUGUUG
UUAUUACCACCAUCUCUUGUUAAUAGCAGCCCAGUAAUAUUUGAUGAA
CAUCUAAUUUGUCCACUGAUGGGAGGGGCAUACAUUGCUUUUCCUACU
UCCUGCCACAUGUUUAUAAAUUGUUUUAUUCUGCAUGGGAGUGUGAUU
GUGUCACUUCCUUCAGUGUUAUUUGACCCUUCAGUACUCCAAGUACUA
UUAAACCAAGUACUAUUAAACAGUUGUGUUGAAUUACAGUAG

or a variant or fragment thereof.

Human T-lymphotropic virus 1 bZIP factor (HBZ) transcript is disclosed by GenBank accession number DQ273132.1 (complete eds) and Satou, Y et al., “HTLV-I basic leucine zipper factor gene mRNA supports proliferation of adult T cell leukemia cells,” PNAS, 103 (3): 720-725 (2006), each of which is herein incorporated by reference in its entirety.

In one embodiment, the viral nucleic acid comprises Human T-lymphotropic virus 1 bZIP factor (HBZ) transcript, or a fragment or variant thereof.

In other embodiments, the viral nucleic acid comprises the nucleotide sequence as set forth in SEQ ID NO: 21:

AGUUGAGCAAGCAGGGUCAGGCAAAGCGUGGAGAGCCGGCUGAGUCUA
GGUAGGCUCCAAGGGAGCGCCGGACAAAGGCCCGGUCUCGACCUGAGC
UUUAAACUUACCUAGACGGCGGACGCAGUUCAGGAGGCACCACAGGCG
GGAGGCGGCAGAACGCGACUCAACCGGCGUGGAUGGCGGCCUCAGGGC
UGUUUCGAUGCUUGCCUGUGUCAUGCCCGGAGGACCUGCUGGUGGAGG
AAUUGGUGGACGGGCUAUUAUCCUUGGAGGAAGAGUUAAAGGACAAGG
AGGAGGAGAAAGCUGUGCUUGACGGUUUGCUAUCCUUAGAAGAGGAAA
GCCGCGGCCGGCUGCGACGGGGCCCUCCAGGGGAGAAAGCGCCACCUC
GCGGGGAAACGCAUCGUGAUCGGCAGCGACGGGCUGAGGAGAAGAGGA
AGCGAAAAAAAGAGCGGGAGAAAGAGGAGGAAAAGCAGAUUGCUGAGU
AUUUGAAAAGGAAGGAAGAGGAGAAGGCACGGCGCAGGAGGCGGGCGG
AGAAGAAGGCCGCUGACGUCGCCAGGAGGAAGCAGGAAGAGCAGGAGC
GCCGUGAGCGCAAGUGGAGACAAGGGGCUGAGAAGGCGAAACAGCAUA
GUGCUAGGAAAGAAAAAAUGCAGGAGUUGGGGAUUGAUGGCUAUACUA
GACAGUUGGAAGGCGAGGUGGAGUCCUUGGAGGCUGAACGGAGGAAGU
UGCUGCAGGAGAAGGAGGAUUUGAUGGGAGAGGUUAAUUAUUGGCAGG
GGAGGCUGGAGGCGAUGUGGUUGCAAUAAUUGCGUGCUUGGUUUACAG
GGAUGACUCAGGGUUUAUAAGAGAGUAAUGGGGGUAUCUGACGCGCGA
GGGGAGGUGUCGUAGCUGACGGAGGAUGCAUGGUCCUGCAAGGAUAAC
AAGAAGGAGUAGCGCGACAAGGGUGAUUCCAGUUUGUAAGGCCUCUCG
AGCCCACUGUGAGAGGCCAAGGUCCCAGUUAAGGCCCCAGCCAGUCAG
GACUCGAUUCUCAAGGGGGGGUCUUUCUUGUAGUAUUGAGACAUGGGA
AUUAGUAAUAUUCAGAAAACAGCACUGUUCUUGUAAUGCUUUGCAUAA
UCCUCCUUGCUCCCAGAACAGGAGAUCAAGGCCUCGUCUGUUCUGGGC
AGCAUACUGCGCAAUUUUGAGUAGAUUUUUGUGGUUUUUGACUAUUGC
UUGAGUUAACUGGGAAAUAUCUUUGUCCACCUCAUGUAGGAGGCUCUU
UCCUGAGGCGAGGGACAUGGAGCCGGUAAUCCCGCCAGCCACUCCGGC
UCCCAUGGCCAGGGCGGAGACAAGCCAGACCGCCACCGGUACCGCUCG
GCGGGAGCGGGAUCCUAGGGUGGGAACAGGUGACAAGGAAAAGGGGGG
CAGGAUGAGGGAGUUAUGACAGGGGGAGGAGACUAUAGCUUGAAUCUG
GGGGUCAAAGCAGUGGGUCCAGUUAAAUGGUAACGUCAGGUGGGGGGC
UGGAAGCGCUAACGAUGGGUAAAGGAGGGGGGUAGAAGAAGAGGAUGG
AACAGAGACGUUGGGAGAGUAUAGGACGUGCCAAGUGGAGAGGCUGGC
ACGAUCGAUACAGACAAUGCAAGUAUAAUUAGUGCUUUGUAGGGUUAA
CUGGACAAGGGUCAGGAGUUUUGAUUUCCAUGGUAUAGAGGGCUCGAG
GAUGUGGUCUAGGUUAGAGUGGGGGAGUAGAGGAGGGGCGGUGGGAGG
CAGUUGGCUGGGUUCGGUAUUAAGGAACCAGAUGGGGUCAUAUCCUGG
AGCGUCGACUAGAAGGGAGAAGGGAAAACCGCAUUUUGAAAAAUGGAG
AUUAAUAUUGAGGCGUGAAACUUCUUGAGUAAAAUUGACAUCGUGCUG
AAACUUCCAGUAGGGGCUGGAGACGGCUCCUGUAUAGGGGCAGGUCCA
UGAUUGGCACCCCAGGUAUGGGCACUUUAAGGAACAAGGGUCUGAAUA
AGAGGCUGAAUAAUAGCCUCCGCCAUUUCGGUUUGGCUUUUUAAUCCA
AUGAGGGAAUAGAUAUAGGGAAUAGGUGGCAUGGUAGCUGGAGUAACU
UACUAGGUUAGGGCAGGGGGGCUGUAGGGCCUGAUCUGCUGAAAGGGC
CAGCAGGUCGAGGGUCCACGAACAAACUGGCUGGGCAGGAUUGCAGGG
UUUAGAGUGGUAUGAGGAGACUCCAAUUGUGAGAGUACAGCAGCUGGG
GCUGUAAUCACCGAAGAUGAGGGGGCAGAACUGGAAGAAUAAAAUCAA
AGUGGCGAGAAACUU

or a variant or fragment thereof.

In other embodiments, the viral nucleic acid comprises the nucleotide sequence as set forth in SEQ ID NO: 22:

AUGGCGGCCUCAGGGCUGUUUCGAUGCUUGCCUGUGUCAUGCCCGGAG
GACCUGCUGGUGGAGGAAUUGGUGGACGGGCUAUUAUCCUUGGAGGAA
GAGUUAAAGGACAAGGAGGAGGAGAAAGCUGUGCUUGACGGUUUGCUA
UCCUUAGAAGAGGAAAGCCGCGGCCGGCUGCGACGGGGCCCUCCAGGG
GAGAAAGCGCCACCUCGCGGGGAAACGCAUCGUGAUCGGCAGCGACGG
GCUGAGGAGAAGAGGAAGCGAAAAAAAGAGCGGGAGAAAGAGGAGGAA
AAGCAGAUUGCUGAGUAUUUGAAAAGGAAGGAAGAGGAGAAGGCACGG
CGCAGGAGGCGGGCGGAGAAGAAGGCCGCUGACGUCGCCAGGAGGAAG
CAGGAAGAGCAGGAGCGCCGUGAGCGCAAGUGGAGACAAGGGGCUGAG
AAGGCGAAACAGCAUAGUGCUAGGAAAGAAAAAAUGCAGGAGUUGGGG
AUUGAUGGCUAUACUAGACAGUUGGAAGGCGAGGUGGAGUCCUUGGAG
GCUGAACGGAGGAAGUUGCUGCAGGAGAAGGAGGAUUUGAUGGGAGAG
GUUAAUUAUUGGCAGGGGAGGCUGGAGGCGAUGUGGUUGCAAUAA

or a variant or fragment thereof.

In some embodiments, the variant sequence comprises least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the sequence as set forth in any one of SEQ ID NOs: 1-22.

The percent sequence identity between two nucleic acid molecules (e.g., between a reference nucleotide sequence and its variant nucleotide sequence) can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the variant sequence differs from the sequence as set forth in any one of SEQ ID NOs: 1-22 by 50, 45, 40, 35, 30, 25, 20, 19, 18, 17, 16, 15, 14, 13, 12, 11, 10, 9, 8, 7, 6, 5, 4, 3, 2, or 1 nucleotide residue(s), when aligned, for example, using any of the previously described alignment methods.

In some embodiments, the variant sequence comprises an alteration at one or more (e.g., several) nucleotide residues of the sequence as set forth in any one of SEQ ID NOs: 1-22, wherein at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more are altered.

In other embodiments, one or more nucleotide residues of the sequence as set forth in any one of SEQ ID NOs: 1-22, or a variant thereof, are glycosylated.

Various motifs and prediction sites of glycosylation of HIV-1 RNA comprising TAR sequence are shown in Table 1.

TABLE 1
Motifst and prediction sites of glycosylation* of HIV-1 RNA.

| SEQ ID | Sequence (5′-3′) |
|---|---|
| 1 | CCAGX₁UX₂X₃GAGCCX₄GGGAGCUCUCUGX₅ (SEQ ID NO: 1), wherein X₁ is A or G; |
| | X₂ is C, U, or A; X₃ is U, A, or C; X₄ is U or C; and X₅ is G or A. |
| 2 | CCAGAUCUGAGCCUGGGAGCUCUCUGG |
| 3 | GGUCUCUCUGGUUAGACCAGAUCUGAGCCUGGGAGCUCUCUGGCUAACUA |
| 4 | GUCUCUCUGGUUAGACCAGAUCUGAGCCUGGGAGCUCUCUGGCUAACUAGG |
| 5 | UCUGGUUAGACCAGAUCUGAGCCUGGGAGCUCUCUGGCUAACUAGGGAACC |
| 6 | AGCUUGCCUUGAGUGCUUCAAGUAGUGUGUGCCCGUCUGUUGUGUGACUC |
| 7 | AGUAGUGUGUGCCCGUCUGUUGUGUGACUCUGGUAACUAGAGAUCCCUCAG |
| 8 | AAAUCUCUAGCAGUGGGCCCGAACAGGGACCUGAAAGCGAAAGGGAAACCA |
| 9 | AUCUCUAGCAGUGGCGCCCGAACAGGGACCUGAAAGCGAAAGGGAAACCAG |
| 10 | AAACCAGAGGAGCUCUCUCGACGCAGGACUCGGCUUGCUGAGCGCGCACGG |
| 11 | GAGGAGCUCUCUCGACGCAGGACUCGGCUUGCUGAAGCGCGCACGGCAAGA |
| 12 | GAGGAGCUCUCUCGACGCAGGACUCGGCUUGCUGAGCGCGCACGGCAAGAG |
| 13 | GGCUUGCUGAGCGCGCACGGCAAGAGGCGAGGGGCGGCGACUGGUGAGUAC |
| 14 | GAGCGCGCACGGCAAGAGGCGAGGGGCGGCGACUGGUGAGUACGCCAAAAA |
| 15 | CGGCAAGAGGCGAGGGGCGGCGACUGGUGAGUACGCCAAAAAUUUUGACUA |
| 16 | UGACUAGCGGAGGCUAGAAGGAGAGAGAUGGGUGCGAGAGCGUCAGUAUUA |
| 17 | AGCGGAGGCUAGAAGGAGAGAGAUGGGUGCGAGAGCGUCAGUAUUAAGCGG |
| 18 | GCUAGAAGGAGAGAGAUGGGUGCGAGAGCGUCAGUAUUAAGCGGGGGAGAA |

†Motifs: Italicized residues (Insulin Like Growth Factor 2 mRNA Binding Protein 1 (IGF2BP1) motif (SEQ ID NOs. 3-7, 10, and 12)); NOP58 motif (SEQ ID NOs. 8-9)); FAM120A motif (SEQ ID NOs.13-18). Feng et al. (2019), Modeling the in vivo specificity of RNA-binding proteins by precisely registering protein-RNA crosslink sites. Mol Cell. 74:1189-1204; Chunmei Cui et al. (2021), GlyinsRNA: a webserver for predicting glycosylation sites on small RNAs, RNA Biology, DOI: 10.1080/15476286.2021.1982574; Naji, S. et al., Host cell interactome of HIV-1 Rev includes RNA helicases involved in multiple facets of virus production,” Mol Cell Proteomics, 11(4) (2012); and rnanut.net/glyinsrna/action.php; each of which is herein incorporated by reference in its entirety.
‡Prediction sites of glycosylation: Underlined residues.

In one embodiment, the viral nucleic acid comprises the nucleotide sequence as set forth in any one of SEQ ID NOs: 1-18, or a variant sequence thereof, wherein at least the underlined residue shown in Table 1 is glycosylated.

In some embodiments, the viral nucleic acid comprises the nucleotide sequence as set forth in any one of SEQ ID NOs: 3-18, or a variant sequence thereof, wherein at least the residue corresponding to position 24, 26, 31, 101, 122, 193, 195, 239, 244, 245, 270, 278, 287, 332, 337, and 344 in SEQ ID NOs: 3-18, respectively, is glycosylated.

In other embodiments, the viral nucleic acids of the present invention, identified as being expressed on the surface of latently infected cells, provide for compositions comprising molecules (e.g., antibodies) which specifically bind to at least the extracellular portion of the viral nucleic acids. In some embodiments, these molecules may be prepared using isolated viral nucleic acid as antigen, followed by screening for reactivity with latently infected cells, or by immunization with an extracellular portion thereof, e.g., alone or in combination with an adjuvant or as a fusion. In various other embodiments, the molecules (e.g., antibodies) can be used as whole molecules, molecule fragments (e.g., antibody fragments), labeled with diagnostic labels, or bound to a cytotoxic agent to kill infected cells. In some embodiments, the viral nucleic acids can be used as antigens to prepare vaccines which are used to immunize a subject having, or is at risk of, a latent viral infection.

In some embodiments, the compositions of the present invention further comprise a pharmaceutically acceptable carrier and/or adjuvant.

Pharmaceutical carriers are known to those skilled in the art. In some embodiments, these would be standard carriers for administration to subject, including solutions such as sterile water, saline, and buffered solutions at physiological pH. Other ingredients include excipients, carriers, thickeners, diluents, buffers, preservatives, and surface active. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's. Preservatives and other additives may also be present such as, for example, antimicrobials, anti-oxidants, chelating agents, and inert gases.

In some embodiments, the pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous, subcutaneous, intraperitoneal or intramuscular injection. In other embodiments, administration may be intracavity or transdermally.

When an antigen is presented to an immune system there are usually many antibodies produced, often recognizing different portions of the antigen. The sera of the antigen-challenged subject may contain all or many of these antibodies, referred to as polyclonal antibodies. Each of the antibodies are produced from a single B cell, which in turn makes many clonal copies of itself. These B cells can be made into hybridomas, which produce a monoclonal antibody. Antibodies specific for nucleic acids are described in e.g., Hu, Z. et al., Expert Rev. Mol. Diagn. 14(7), 895-916 (2014) and Ye, J., et al., PNAS, 105:82-87 (2008), each of which is herein incorporated by reference in its entirety.

In some embodiments, the composition of the present invention comprises the molecule that specifically binds the extracellular portion of the viral nucleic acid present on the plasma membrane of the host cell infected with the virus, wherein the molecule comprises an antibody or aptamer.

In other embodiments, the molecule comprises an antibody that specifically binds the extracellular portion of the viral nucleic acid present on the plasma membrane of the host cell infected with the virus.

The antigens that are determinative of the latent stage of a viral infection can contain the entire viral nucleic acid as the native epitope, or the extracellular portion thereof, sufficient to react with antibody and/or elicit an immune response. An antibody to an antigen of choice can be produced in mice according to Kohler and Milstein, Nature. 256:495-497 (1975) and Eur. J. Immunol. 6:51 1-519 (1976), by immunizing a host with the antigen of choice. Once a host is immunized with the antigen, B-lymphocytes that recognize the antigen are stimulated to grow and produce antibody to the antigen. Each activated B-cell produces clones which in turn produce the monoclonal antibody. Hybridomas may be produced using e.g., the methods developed by Kohler and Milstein, Nature, 256:495-497 (1975). The antibodies produced and isolated by this method are specific for a single antigen or epitope on an antigen, and are referred to as monoclonal antibodies. A cell bound enzyme linked immunosorbent assay (ELISA) can be used to screen supernatants from growing hybridomas (Glassy, M. C. and Surh, C. D., J. Immunol. Method. 81:115 (1985)). Cells which bind the antibody or produce the antibody can be analyzed using flow cytometry.

In some embodiments, chimeric antibodies can be used. Chimeric antibodies are antibodies in which the various domains of the antibodies' heavy and light chains are coded for by DNA from more than one species. For example, a chimeric antibody can comprise the variable domains of the heavy (VH) and light (VL) chains derived from the donor species producing the antibody of desired antigenic specificity, and the variable domains of the heavy (CH) and light (CL) chains derived from the host recipient species. Thus, for example, in some embodiments, it is possible to produce a chimeric antibody for in vivo clinical use in humans which comprises mouse VH and VL domains coded for by DNA isolated from a rabbit that binds the extracellular portion of the viral nucleic acid present on the plasma membrane of the host cell infected with the virus and CH and CL domains coded for with DNA isolated from a human immune system cell. Under certain circumstances, monoclonal antibodies of one isotype might be more preferable than those of another in terms of their diagnostic or therapeutic efficacy. For example, in other embodiments, unmodified mouse monoclonal antibodies of isotype gamma-2a or gamma-3 may be used for lysing target host cells infected with the virus.

In another embodiment, the antibody or antibody fragment, such as, for example, Fab and F(ab′), as well as isotypes, particularly if labeled or bound to a cytotoxic agent, can be used since having an effect on the viral infection in these situations is not dependent upon complement-mediated cytolytic destruction of those cells bearing the latent viral protein.

One of ordinary skill in the art readily knows how to apply the methods and techniques discussed herein to compositions that do not include an antibody per se, but have antigen recognition properties.

In another embodiment, the molecule comprises an aptamer that specifically binds the extracellular portion of the viral nucleic acid present on the plasma membrane of the host cell infected with the virus.

In vitro selection of oligonucleotides carried out against RNA structures have led to aptamers that differ from antisense sequences in that they are not directed against a linear nucleic acid sequence but which take into account local non-linear structures, in particular the three-dimensional conformations of the target RNA (e.g., hairpins, pseudo-knots, and the like). International Patent Publication No. WO1996/004374 to Jean-Jacques Toulmé et al., which is herein incorporated by reference in its entirety, describes oligonucleotide recognizing a non-linear nucleic acid sequence, therapeutic applications thereof and method for preparing same. For example, WO1996/004374 describes a method of preparation by in vitro selection of an oligonucleotide, characterized in that: a) a population of candidate oligonucleotides of random sequences is prepared, b) this population of candidate oligonucleotides is put in the presence of the target sequence, the candidates being in excess with respect to the target sequence, and c) the oligonucleotides which form a stable complex with the target sequence are isolated, and d) the nucleic acid samples obtained are amplified at 1 step c), and c) steps b) and c) are repeated several times. Watrin, M. et al., “Aptamers targeting RNA molecules,” Methods Mol Biol., 535:79-105 (2009), which is herein incorporated by reference in its entirety, describe a genomic SELEX approach and its application to the recognition of stem-loop structures prone to the formation of kissing complexes. Watrin, M. et al. also provide technical details for running a procedure termed 2D-SELEX that takes advantage of both in vitro selection and dynamic combinatorial chemistry that allows selecting aptamer derivatives containing modified nucleotides that cannot be incorporated by polymerases.

In some embodiments, the molecule that specifically binds the extracellular portion of the viral nucleic comprises an oligonucleotide that specifically binds the extracellular portion, wherein the oligonucleotide recognizes and specifically binds a non-linear structure of the extracellular portion.

In other embodiments, the molecule comprises an oligonucleotide that specifically binds the extracellular portion, wherein at least a segment of the oligonucleotide is complementary to at least a segment of the extracellular portion of the viral nucleic acid.

In another embodiment, the molecule comprises a peptide that specifically binds the extracellular portion of the viral nucleic acid present on the plasma membrane of the host cell infected with the virus. Walker, M. J. & Varani, G., “Structure-Based Design of RNA-Binding Peptides,” Methods Enzymol., 623:339-372 (2019), which is herein incorporated by reference in its entirety, describe engineering peptides that bind to structured RNAs by highlighting methods and design strategies. In some embodiments, peptides binding the extracellular portion of the viral nucleic acid, which are developed de novo using combinatorial methods, can be used.

In other embodiments, the molecule (e.g., aptamers, peptides) that specifically binds the extracellular portion of the viral nucleic acid present on the plasma membrane of the host cell infected with the virus is coupled to a radioisotope or toxin, for example, for delivery to the target cell, as described with respect to the antibodies.

There are a number of uses for the compositions disclosed herein, for example, in diagnostic assays and kits, vaccine preparations, therapeutics, and/or prophylaxis. For example, the destruction of cells harboring latent virus reduces the effects of the virus on the host and treat or prevent any subsequent diseases that are dependent on the presence of the virus. For example, diseases such as acquired immunodeficiency syndrome (AIDS) (or any of the other conditions or diseases associated with HIV e.g., HIV-Associated Neurocognitive Disorder (HAND), stroke, autoimmunity, cancer and premature aging, could thereby be treated or prevented by the elimination or reduction in HIV infected cells.

In one embodiment, the molecule that specifically binds the extracellular portion of the viral nucleic acid present on the plasma membrane of the host cell infected with the virus comprises a diagnostic or therapeutic moiety.

In some embodiments, the molecules (e.g., aptamers, antibodies or fragments thereof) can be used to identify, remove and/or kill latently infected cells (e.g., human host cells latently infected with HIV). In one embodiment, the molecules (e.g., aptamers, antibodies or fragments thereof) will be coupled to a label which is detectable or cytotoxic but which does not interfere with binding to the infected cells. Examples include radioisotopes, such as indium (“In”), which is useful for diagnostic purposes, and yttrium (“Y”), which is cytotoxic. Other detectable labels include enzymes, radioisotopes, fluorescent compounds, chemiluminescent compounds, and bioluminescent compounds and compounds detectable by ultrasound, MRI or CT. Other cytotoxic agents include toxins such as ricin, mitomycin C, danorubicin, and vinblastin.

In other embodiments, the diagnostic or cytotoxic agents can be coupled either directly or indirectly to the molecules (e.g., aptamers, antibodies or fragments thereof). In one embodiment, indirect coupling is via a spacer moiety. These spacer moieties, in turn, can be either insoluble or soluble and can be selected to enable release of the agent from the molecules (e.g., aptamers, antibodies or fragments thereof) at the target site.

In another embodiment, radioisotopes can be attached directly to the molecules (e.g., aptamers, antibodies or fragments thereof); and, in other embodiments, others require an indirect form of attachment. The radioisotopes iodine-125 (¹²⁵I), iodine-131 (¹³¹I), technetium-99m (^{99m}Tc), rhenium-186 (¹⁸⁶Re) and rhenium-188 (¹⁸⁸Re) can be covalently bound to proteins (including antibodies) through amino acid functional groups; and for radioactive iodine it is usually through the phenolic group found on tyrosine. There are numerous methods known in the art to accomplish coupling, including but not limited to: chloramine-T (Greenwood, F. C. et al., Biochem J. 89:114-123 (1963)); Iodogen method (Salacinski, P. et al., Anal. Biochem. 117:136-146 (1981)); and Tc and Re can be covalently bound through the sulfhydryl group of cysteine (Griffiths, G. L. et al., Cancer Res., 51:4594-4602 (1991)).

In other embodiments, for in vivo diagnosis, radioisotopes may be bound to the molecule that specifically binds the extracellular portion of the viral nucleic acid either directly or indirectly by using an intermediate functional group such as, but not limited to, bifunctional chelating agents such as diethylenetriaminepentacetic acid (DTPA) and ethylenediaminetetraacetic acid (EDTA) and similar molecules. The molecule that specifically binds the extracellular portion molecule (for example, the antibody), and the agent can be linked in several ways. In one embodiment, if a fusion is produced by expression of a fused gene, a peptide bond serves as the link between e.g., the cytotoxin and the molecule that specifically binds the extracellular portion of the viral nucleic acid.

Alternatively, in another embodiment, the toxin or other moiety and the molecule that specifically binds the extracellular portion of the viral nucleic acid can be produced separately and then subsequently coupled via a non-peptide covalent bond. For example, in some embodiments, the covalent linkage can be a disulfide bond (e.g., a DNA encoding the antibody or peptide that specifically binds the extracellular portion of the viral nucleic acid can be engineered to contain an extra cysteine codon and e.g., the toxin or other moiety can be derivatized with a sulfhydryl group reactive with the cysteine of the modified antibody or peptide). In other embodiments, in the case of a peptide toxin or other moiety, this can be accomplished by inserting a cysteine codon into the DNA sequence encoding the toxin or other moiety. In still further embodiments, a sulfhydryl group, either by itself or as part of a cysteine residue, can be introduced using solid phase polypeptide techniques known in the art. For example, once the correct sulfhydryl groups are present, the toxin or other moiety and antibody or peptide can be purified, both sulfur groups reduced, mixed, and disulfide bond formation allowed to proceed to completion. The mixture can then be dialyzed or chromatographed to remove unreacted entities.

Numerous types of cytotoxic compounds can be joined to proteins through the use of a reactive group on the cytotoxic compound or through the use of a cross-linking agent. A common reactive group that will form a stable covalent bond in vivo with an amine is isothiocyanate. This group preferentially reacts with the ϵ-amine group of lysine. Maleimide is a commonly used reactive group to form a stable in vivo covalent bond with the sulfhydryl group on cysteine. In some embodiments, radiometal ions can be attached to monoclonal antibodies indirectly through the use of chelating agents that are covalently linked to the antibodies. Chelating agents can be attached through amines and sulfhydral groups of amino acid residues and also through carbohydrate groups. Examples of crosslinking agents include bismaleimidohexane (BMH), which is reactive with sulfhydryl groups, ethylene glycolbis[succinimidylsucciate] (EGS), which is reactive with amino groups, and maleimidobenzoyl-N-hydroxysuccinimide ester (MBS). These methodologies are routine and known in the art.

In one embodiment, toxins and substances which elicit the host to attack the virally infected cells, as well as synthetic or natural chemotherapeutic drugs, and/or radioactive colloids, can be conjugated to the molecules (e.g., aptamers, antibodies or fragments thereof) that specifically bind the extracellular portion of the viral nucleic acid using standard chemical techniques, or in some cases, using recombinant technology, for example, as fusion proteins. The molecules (e.g., aptamers, antibodies or fragments thereof) can also be coupled to a signal protein that induces apoptosis (or programmed cell death).

For example, diphtheria toxin is a substance produced by Corynebacterium diphtheria that can be used therapeutically. In some embodiment, the toxic alpha component can be bound to the molecules (e.g., aptamers, antibodies or fragments thereof) that specifically bind the extracellular portion of the viral nucleic acid and used for site-specific targeting to a cell harboring a latent virus (e.g., HIV). Lectins are proteins that bind to specific sugar moieties. Ricin is a toxic lectin which has been used immunotherapeutically. In some embodiments, this is accomplished by binding the alpha-peptide chain of ricin, which is responsible for toxicity, to the molecules (e.g., aptamers, antibodies or fragments thereof) to enable site specific delivery of the toxic effect. Other useful toxins include cholera toxin, Shiga-like toxin (SLT-I, SLT-II, SLT-IIV), LT toxin, C3 toxin, Shiga toxin, pertussis toxin, tetanus toxin, Pseudomonas exotoxin, alorin, saporin, modeccin, and gelanin.

Radioisotopes can be used as diagnostics and followed after administration using standard non-invasive radio-imaging techniques. As radioisotopes decay, they emit characteristic photons or particles or both. Photons, commonly referred to as gamma rays, are penetrating. If their energy level is high enough, they can travel through the body and be detected by diagnostic instrumentation. Radioisotopes that emit photons can be attached to the molecules (e.g., aptamers, antibodies or fragments thereof) that specifically bind the extracellular portion of the viral nucleic acid and used for diagnostic imaging (e.g., radioimmunoscintigraphy (RIS)). The shorter the distance between the antigen and the target, the shorter the required range of emission of the radioisotope. Auger electrons have a very short path length (5-10 nm) and need to be internalized to be cytotoxic. In such embodiments, only the molecules (e.g., aptamers, antibodies or fragments thereof) that specifically bind the extracellular portion of the viral nucleic acid and are internalized after binding to the cell should be considered for radioisotopes that emit Auger electrons. Alpha particles need to be close to a cell (within 3-4 cell diameters) to be effective. Both Auger electrons and alpha emitters have high selectivity because their short-range emission will not irradiate neighboring virally uninfected cells.

In other embodiments, the molecules (e.g., aptamers, antibodies or binding fragments thereof) that specifically bind the extracellular portion of the viral nucleic acid can also be labeled with a paramagnetic isotope for purposes of in vivo diagnosis, as in magnetic resonance imaging (MRI) or electron spin resonance (ESR). In general, any conventional method for visualizing diagnostic imaging can be utilized.

In some embodiments, the compositions of the present invention can be used to deliver specific cargo (e.g., drugs, toxins, gene editing constructs) into infected cells.

In one embodiment, the composition of the present invention comprises the molecule that specifically binds the extracellular portion of the viral nucleic acid present on the plasma membrane of the host cell infected with the virus, wherein the composition is an exosome, a liposome, a macrovesicle, or a nanoparticle that is coated with the molecule that specifically binds the extracellular portion of the viral nucleic acid.

In another embodiment, the composition is an extracellular vesicle (EV), wherein the EV is coated with the molecule that specifically binds the extracellular portion of the viral nucleic acid.

In other embodiments, the composition is a colloidal dispersion system, such as a macromolecule complex, a nanocapsule, a nanoparticle, a microsphere, a bead, or a lipid-based system including oil-in-water emulsions, micelles, mixed micelles, and liposomes, wherein the colloidal dispersion or lipid-based system is coated with the molecule that specifically binds the extracellular portion of the viral nucleic acid.

U.S. Patent Publication No. 2017/0349914 to David Benjamin Turitz Cox et al., which is herein incorporated by reference in its entirety, describes delivery systems targeting cells including exosomes and colloidal dispersion or lipid-based system. U.S. Patent Publication No. 2020/0109183 to Wiklander, O. et al., which is herein incorporated by reference in its entirety, describes methods for coating of EVs as well as pharmaceutical compositions and medical applications of such EVs coated with Fc containing proteins.

In still further embodiments, the exosome, the liposome, the macrovesicle, or the nanoparticle that is coated with the molecule that specifically binds the extracellular portion of the viral nucleic acid further comprises one or more elements of a gene editing system.

In one embodiment, the gene editing system comprises a CRISPR-Cas system, a designer zinc finger (ZFN) system, transcription activator-like effectors (TALEs), or homing meganucleases.

In another embodiment, the gene editing system comprises a CRISPR-Cas system.

In one embodiment, the CRISPR/Cas system is a type I, II, or III system. Non-limiting examples of suitable CRISPR/Cas proteins include Cas3, Cas4, Cas5, Cas5e, Cas6f, Cas7, Cas8a1, Cas8a2, Cas8b, Cas8c, Cas9, Cas10, Cas10d, CasA, CasB, CasC, CasD, CasE, CasF, CasG, CasH, Csy1, Csy2, Csy3, Csc1, Csc2, Csa5, Csn2, Csm2, Csm3, Csm4, Csm5, Csm, Cmri, Cmr3, Cmr4, Cmr5, Csbi, Csb2, Csb3, Csx17, Csx14, Csx10, Csx16, CsaX, Csx3, Cszl, Csx15, Csfi, Csf2, Csf3, and Csf4.

In another embodiment, the targeting nucleotide sequence of the gRNA is designed to bind or hybridize to the target sequence of the viral (e.g., HIV-1) genome, and thus the targeting nucleotide sequence of the gRNA is complementary or substantially complementary to the target sequence of the viral (e.g., HIV-1) genome. In other embodiments, the gRNA comprises a targeting nucleotide sequence that is directed to a target site in the viral (e.g., HIV-1) genome. For example, the viral (e.g., HIV-1) genome may comprise one or more target sequences present in the sense or antisense strand. The target sequence in the viral (e.g., HIV-1) genome may be any sequence in any coding or non-coding region.

In some embodiments, the target sequence is present in a long terminal repeat (LTR) region of the virus e.g., an enhancer and/or core promoter region of HIV (e.g., HIV-1 or HIV-2) LTR.

In other embodiments, the target sequence in the viral (e.g., HIV-1) genome may be any sequence in any LTR region where gRNA-mediated truncation would result in truncated LTR DNA in the infected host cells.

U.S. Patent Publication No. 2018/0073018 to HU, W. et al., which is herein incorporated by reference in its entirety, describes compositions and methods for reactivation of HIV in latently infected cells using a CRISPR/Cas system in combination with an activation system to provide enhanced transcriptional activation of the HIV genome, and once reactivated, HIV may be cleared by the host immune system or using any viral clearance methodology known in the art.

In another embodiment, the target sequence in the viral (e.g., HIV-1) genome may be any sequence in any coding or non-coding region where gRNA-mediated localization of one or more transcription activators would result in increased activation/reactivation of the transcription of the HIV genome, or portion thereof.

In some aspects, the present invention provides the molecule (e.g., antibody, aptamer) that specifically binds the extracellular portion of the viral nucleic acid present on the plasma membrane of the host cell infected with the virus, as disclosed herein. In some embodiments, the molecule is an antibody or aptamer as disclosed herein.

In still further aspects, the present invention provides the exosome, the liposome, the macrovesicle, or the nanoparticle that is coated with the molecule that specifically binds the extracellular portion of the viral nucleic acid, as disclosed herein. In some embodiments, the exosome, the liposome, the macrovesicle, or the nanoparticle that is coated with the molecule further comprises one or more elements of a gene editing system. In one embodiment, the molecule is an antibody or aptamer as disclosed herein. In another embodiment, the gene editing system is a CRISPR/Cas system. In some embodiment, the exosome coated with the molecule that specifically binds the extracellular portion of the viral nucleic acid can specifically deliver the cargo (e.g., gene editing system) to the host cell latently infected with the virus.

In other aspects, the present invention provides a method for treating or preventing infection by a virus. The method comprises administering a composition of the present invention as disclosed herein to a subject in an amount effective to treat or prevent the infection.

Accordingly, in some embodiments, the present invention provides methods of preventing, treating and/or managing a viral infection (e.g., a latent viral infection (e.g., latent HIV infection)), said methods comprising administering to a subject in need thereof one or more compositions and/or components (e.g., immunogenic compositions, antibodies, aptamers, exosomes) disclosed herein. In other embodiments, said method of preventing, treating and/or managing the viral infection comprises administering to a subject in need thereof one or more doses of a prophylactically or therapeutically effective amount of one or more compositions and/or components (e.g., immunogenic compositions, antibodies, aptamers, exosomes) disclosed herein.

In one embodiment, the composition comprises the extracellular portion of the viral nucleic acid present on the plasma membrane of the host cell infected with the virus.

In another embodiment, the composition is an immunogenic composition (e.g. vaccine) to induce a reaction to the viral nucleic acid present on the plasma membrane of the host cell infected with the virus, alone or in combination with an adjuvant. Numerous vaccine formulations are known to one of ordinary skill in the art.

In some embodiments, the viral nucleic acid, or the extracellular portion thereof, can be obtained by isolating the naturally occurring viral nucleic acid, or the extracellular portion thereof; or in other embodiments, engineered viral nucleic acid, or extracellular portion thereof, can either be made through synthetic mechanisms or through recombinant biotechnology techniques. The viral nucleic acid, or the extracellular portion thereof, can be synthesized using any one of the methods known to those skilled in the art. In other embodiments, immunogenic fusion derivatives can be made by fusing a moiety sufficiently large to confer or enhance immunogenicity.

In another embodiment, the composition comprises the molecule that specifically binds the extracellular portion of the viral nucleic acid present on the plasma membrane of the host cell infected with the virus.

In some embodiments, the composition is administered to the subject during a latent life cycle of the virus.

In other embodiments, the subject has tested positive for HIV infection but has no symptoms associated with HIV.

In another embodiment, the subject has acquired immunodeficiency syndrome (AIDS).

In one embodiment, the methods of the invention provide for treatment or prevention of diseases and disorders associated with viral (e.g., HIV) infection.

In some embodiments, administration and/or dosages of the compositions of the present invention can be suitable for inducing an immune response to the infected cell, killing of the infected cells, identifying infected cells and/or removal of infected cells. In some embodiments, the molecule (e.g., antibody, aptamer) conjugated to a cytotoxic agent can also be used for immunotherapy to kill cells infected with a virus that is at least partly in the latent stage of its life cycle and is expressing the viral nucleic acid of latency that is reactive with the disclosed molecules (e.g., antibodies, aptamers) of the invention. Effective dosages of such conjugates will vary based upon affinity, selectivity and concentration of the molecule.

In others embodiments, the dosage of a composition of the present invention can vary with the age, condition, and extent of the viral infection or disease in the subject and can be determined by one of ordinary skill in the art. The dosage can be adjusted by e.g., a physician and/or other suitable health care professionals. Dosage can vary from one to multiple doses administered daily, for one to several days.

In some embodiments, a protocol for vaccination to induce an immune response to the infected cells can be designed based on standard techniques. Efficacy can be determined based on measurements of antibody titers to the latent viral nucleic acid and using diagnostic techniques to determine the presence and/or number of infected cells remaining after treatment. In one embodiment, vaccination protocols range from a single immunization to multiple boosters. In other embodiments, priming vaccinations followed by boosters may be required, or it may be necessary to utilize a prolonged treatment protocol with low quantities of antigen.

In another aspect, the present invention provides a method for identifying a subject infected with a virus. The method comprises detecting the presence of an extracellular portion of a viral nucleic acid present on a plasma membrane of a host cell infected with a virus using the composition of the present invention as disclosed herein.

In some embodiments, the detecting step is during a latent life cycle of the virus.

In other embodiments, the host cell infected with the virus is from a subject having tested positive for HIV infection but has no symptoms associated with HIV.

In one embodiment, the host cell infected with the virus is from a subject having acquired immunodeficiency syndrome (AIDS).

In other embodiments, labeled molecule (e.g., antibody, aptamer) conjugates can be used to confirm and/or quantitate the presence of a latent viral infection. In some embodiments, the labelled molecule (e.g., antibody, aptamer) conjugates can be used to determine whether a particular therapeutic regimen aimed at reducing the viral infection is effective.

In one embodiment, the labeled molecules (e.g., antibody, aptamer) specifically binding to the extracellular portion of the viral nucleic acid present on the plasma membrane of the host cell infected with the virus can be used in assays with either liquid phase or solid phase carriers and with any of a variety of labels, in either competitive or noncompetitive assays and in either a direct or indirect format. Examples of such immunoassays are the radioimmunoassay (RIA) and the sandwich (immunometric) assay. Immunoassays can be run in either the forward, reverse, or simultaneous modes, including immunohistochemical assays on physiological samples.

The extracellular portion of the targeted viral nucleic acid is presented on the surfaces of cells. As such, in another embodiment, these cells can be detected by the disclosed molecules (e.g., antibody, aptamer) in biological fluids and tissues. Any sample containing a detectable amount of the extracellular portion of the viral nucleic acid or a cell expressing the extracellular portion of the viral nucleic acid can be used. In some embodiments, a sample is a liquid sample such as saliva, cerebrospinal fluid, blood, or serum or a solid or semi-solid such as tissues.

In some embodiment, the disclosed labeled molecule (e.g., antibody, aptamer) can be used for in vivo detection, wherein the detectably labeled molecule is administered to the subject in a dose sufficient to detect cells latently infected with virus. In other embodiments, in vivo diagnostic imaging is performed using a radioisotope as the label for the labeled molecule (e.g., antibody, aptamer) of the present invention.

In other aspects, the present invention provides a method for separating or killing virally infected cells ex vivo, the method comprising contacting cells obtained from a subject with a composition as disclosed herein, wherein the virally infected cells bind to the molecule that specifically binds the extracellular portion.

In one embodiment, virally infected cells (e.g., latently infected cells), once identified by the compositions disclosed herein, can be separated from the remainder of the cells (e.g., uninfected cells) using a selective process employing the molecule that specifically binds the extracellular portion as disclosed herein.

Fluorescence-activated cell sorting (FACS) separates a population of cells into sub-populations based on fluorescent labeling. For example, cells stained using fluorophore-conjugated antibodies can be separated from one another depending on which fluorophore they have been stained with. For example, a host cell infected with the virus and expressing the viral nucleic acid on the plasma membrane may be detected and separated from other cells using e.g., an FITC- or PE-conjugated antibody that recognizes the extracellular portion of the viral nucleic acid present on the plasma membrane of the host cell infected with the virus.

In another embodiment, the contacting step comprises FACS.

In other embodiments, the molecule (e.g., antibody, aptamer) that binds the extracellular portion of the viral nucleic acid present on the plasma membrane of the host cell infected with the virus can be covalently bound to a solid phase and employed as a composition for separation of latently infected cells from the remainder of cells. In one embodiment, the subject's blood can be passed through an extracorporeal reactor or filter having the molecule (e.g., antibody, aptamer) immobilized thereon or therein.

In one embodiment, the cells are obtained from the subject during a latent life cycle of the virus. In another embodiment, the subject has tested positive for HIV infection but has no symptoms associated with HIV. In other embodiments, the subject has acquired immunodeficiency syndrome (AIDS).

In some embodiments, the infected cells are in a transfusion. In other embodiments, the infected cells are in a bone marrow or solid organ transplant.

In other aspects, the present invention provides a kit for use with methods and compositions disclosed herein. Compositions, probes, antibodies, aptamers, labels, and the like as disclosed herein may be provided in the kit. The kits can also include a suitable container and optionally one or more additional agents. In some embodiments, the container is a vial, test tube, flask, bottle, syringe holder and/or other container(s). In other embodiments, the kit comprises one or more compositions of the present invention disclosed herein, a pharmaceutically acceptable carrier, an applicator, and/or instructional material for use thereof.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific aspects and embodiments thereof, it should be understood by those skilled in the art that various changes may be made and equivalents may be substituted without departing from the true spirit and scope of the invention. It will be readily apparent to those skilled in the art that other suitable modifications and adaptations of the compositions and methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The embodiments are now described with reference to the following Examples. These Examples are provided for the purpose of illustration only, and the embodiments are not limited to these Examples, but rather encompasses all variations that are evident as a result of the teachings provided herein.

### Example 1

### Materials and Methods

The following materials and methods are applicable to the disclosed examples.

### Cell Culture and Reagents

Jurkat (uninfected T-lymphocytes), J1.1 (HIV-1-infected T-lymphocytes), U1 (HIV-1-infected promonocytic) and U937 (promonocytic) cells were cultured in complete RPMI 1640 media with 10% fetal bovine serum (FBS), 1% L-glutamine, and 1% penicillin/streptomycin (Quality Biological, Gaithersburg, MD, USA). Infected cultured central memory CD4 T cells were generated as previously described until day 13 (Macedo, A. B. et al., “Influence of Biological Sex, Age, and HIV Status in an In Vitro Primary Cell Model of HIV Latency Using a CXCR4 Tropic Virus,” AIDS Res. Hum. Retroviruses, 34:769-777 (2018) and was generous gift from Dr. Alberto Bosche (GWU, USA).

A set of primary PBMCs were (Precision For Medicine, Frederick, MD, USA) cultured in vitro first in PMA/GM-CSF for 3 days to obtain monocyte derived macrophages. Cells were then infected with HIV-1 89.6 strain (MOI: 10) for 3 days. Cells were treated with PMA/IL-2. On Day 3 post infection, cells were treated with PMA/IL-7 and a cART cocktail (equal parts of lamivudine (NRTI), tenofovir disoproxil fumarate (NtRTI), emtricitabine (NRTI), and indinavir (protease inhibitor) at 10 μM each). The cART/IL-7 treatment was repeated every other day for the course of one week and cells were harvested for RT-qPCR.

### RNA Isolation and Quantitative Real-Time PCR (RT-qPCR)

For the isolation of total RNA, cells were harvested, washed once in 1×PBS without calcium or magnesium and resuspended in 50 μL of 1×PBS. In this study, total RNA was isolated from the plasma membrane mostly and from Cytoplasm, Nuclear and extracellular vesicles (EVs) in some parts of the study using Trizol Reagent (Invitrogen) as described by the manufacturer's protocol. For RNA estimation, cDNA was generated using GoScript Reverse Transcription Systems (Promega) using Reverse Primers shown in Table 2.

TABLE 2
Reverse Primers.

| SEQ ID | RNA Identity | Sequence for RT Reaction (5′-3′) |
|---|---|---|
| 23 | TAR | CAA CAG ACG GGC ACA CAC TAC (99-119) |
| 24 | TAR1 | CAA CAG ACG GGC ACA CAC TAC (161-184) |
| 25 | TAR2 | CCACTGCTAGAGATTTTCCACACT (161-184) |
| 26 | TAR3 | CCCCCTGGCCTTAACCGAATTTT (387-409) |
| 27 | TAR4 | TATCTCTATCCTTTGATGCACACAA (593-617) |
| 28 | Long LTR | GGGCGCCACTGCTAGAGA (626-643) |
| 29 | Pol | CAAATTTCTACTAATGCTTTTATTTTTTC (2634-2662) |
| 30 | env | TGGGATAAGGGTCTGAAACG (7917-7936) |
| 31 | nef | TGTAAGTCATTGGTCTTAAAGGTACCTGAGG (9010-9040) |

RT-qPCR conditions were as follows: one cycle for 2 min at 95° C. followed by 41 cycles of 95° C. for 15 s and 58° C. for 40 s.

Serial dilutions of DNA from a CEM T-cell line containing a single copy of HIV-1 LAV provirus per cell (8E5 cells) were used as the quantitative standards. The cDNA samples (2 μL per well) for each RNA type were plated into a Master Mix (18 μL per well) containing IQ Supermix (Bio-Rad), TAR Forward Primer (SEQ ID NO: 32: 5′-GGT CTC TCT GGT TAG ACC AGA TCT G-3′, 1-25), TAR Reverse Primer (SE ID NO: 33: 5′-CAA CAG ACG GGC ACA CAC TAC-3′ 76-96), and TAR Probe (SEQ ID NO: 34: 5′56-FAM-AG CCT CAA TAA AGC TTG CCT TGA GTG CTT C-36-TAMSp-3′). Reactions were performed in triplicate using the BioRad CFX96 Real-Time System. Quantitation was determined using cycle threshold (Ct) values relative to the 8E5 standard curve using the BioRad CFX Manager Software. All reactions were run in triplicate on the CFX96 Real-Time PCR Detection System (Bio-Rad, Hercules, California 94547, USA) and analysis of generated raw data was analyzed using Microsoft Excel 2016.

### Transfection of HIV-1 DNA

The Cell-Porator™ [Life Technologies, Inc; Bethesda Research Labs (BRL)] was used to transfect the Myeloids (U937) and T-cells (CEM) per the manufacturer's instructions. Briefly, cells (5×10⁶ cells per sample) were electroporated in RPMI media containing 10% FBS and 5% L-glutamine. The cell lines were transfected with pNL4-3 DNA (20 μg) at the following parameters: a capacitance of 800 μF, low resistance, pulse voltage of 230 V and fast charge rate.

### Preparation of Whole Cell Extracts

Jurkat, J1.1, U1, U937 and CEM cells were centrifuged at 1800×RPM at room temperature for 5 min. Cell pellets were washed with 1× Phosphate Buffer Saline (PBS) without Ca++ and Mg++ and resuspended in lysis buffer comprising 120 mM NaCl, 50 mM Tris-HCl, pH 7.5, 50 mM NaF, 5 mM EDTA, 0.5% NP-40 (NP40), 0.2 mM Na₃VO₄, 1 mM DTT and 1 complete protease inhibitor cocktail tablet/50 mL (Roche Applied Science, Mannheim, Germany). Cell pellets were incubated on ice for 20 min with vortexing every 5 min. The whole cell lysate was separated from cell debris by centrifugation at 10,000×RPM at 4° C. for 10 min. Bradford assay (Bio-Rad) was used to determine the protein concentration according to the manufacturer's protocol.

### Western Blot Analysis and Antibodies

Laemmli buffer (Tris Glycine SDS and β-mercaptoethanol) was added to whole cell lysate samples. Samples were heated at 95° C. for 3 min, then loaded into 4-20% Tris-glycine gels (Invitrogen) with a Precision Plus Protein™ Standard (BioRad) and fractionated at 150 V. Gels were transferred onto Immobilon Polyvinylidene fluoride (PVDF) membranes (Millipore) at 50 milliamps overnight. Five percent milk in PBS containing 0.1% Tween-20 (PBS-T) was used to block the protein membranes for 2 h at 4° C. Primary antibody in PBS-T; HSP90 (Novus Biologicals, NB100-1972) was added to the membranes prior to overnight incubation at 4° C.; primary antibodies include α-p24 were obtained from NIH AIDS Reagent Program, Manassas, VA, USA. Membranes were washed three times with PBS-T (5 min/wash). Complementary HRP-conjugated secondary antibodies were added, followed by incubation for 2 h at 4° C. Membranes were washed twice with PBS-T and once with PBS, 5 min per wash. HRP luminescence was activated with Clarity Western ECL Substrate (Bio-Rad). Membranes were developed using the Molecular Imager ChemiDoc Touch system (Bio-Rad).

### Plasma Membrane Extraction

Cells were cultured in growth media and spun down at 600×g for 5 min at 4° C. Cell pellets were washed with ice-cold 1× Phosphate Buffer Saline (PBS) without Ca++ and Mg++. Plasma membranes were isolated using the Plasma Membrane Protein Extraction Kit (ab65400, Abcam) from Abcam. Briefly, Cell pellets were resuspended in 2 mL of Homogenize Buffer (HB) Mix. Cell suspension was Dounce Homogenized on ice for 50-60 strokes and then, spun at 700×g for 10 min at 4° C. This pellet was the nuclear fraction and supernatants were transferred to new tubes and spun again at 10,000×g for 30 min at 4° C. The pellets generated from this spin were crude membranes and the supernatant was soluble cytosol.

### Immunoprecipitation

Five micrograms of each IgG and J2 (RNA binding antibody; Scicons; Cat #J2) were used for immunoprecipitation. In another reaction, 1 μg of Human rec-hSiglec-14 (R&D Systems; cat #4905-SL-050) was added to the samples (overnight) prior to immunoprecipitation with Siglec-5/Siglec-14 antibody (5 ug, Biorad; cat #MCA5787GA). Protein A/G beads were added the next day (2 hours, at 4° C.) and the IPed samples were washed. RNA was isolated for RT-qPCR using TAR primers. An IP with IgG antibody served as a control.

### Sialidase Enzymatic Digestion

To digest the Glycans, 0.8-1 ug of RNA was incubated with 1 ul of Sialidase enzyme (New England Biolabs (NEB) Cat #P0722) in GlycoBuffer 1 (10×) and reaction was incubated at 37° C. for 1 hour. After one hour, enzyme was heat deactivated by keeping the sample at 65° C. for 10 minutes and processed samples were used for downstream RT/qPCR reactions.

### Flow Cytometry

1×10⁶ cells from each cell type were centrifuged, washed and fixed with 70% alcohol and Anti-Fc antibody dilution was used to prevent nonspecific binding of primary antibody, stained using a primary antibody against dsRNA (anti-dsRNA J2) followed by a donkey anti-mouse Alexa-488 secondary antibody and analyzed using a BD FACSaria II Flow Cytometer.

### Example 2

### Presence of HIV-1 TAR RNA in the Membrane of Infected Cells

Multiple assays were used to detect and validate presence of HIV-1 TAR only (not genomic RNA) on the membrane. HIV-1 TAR RNA is a 59-nucleotide long non-coding RNA positioned in the 5′-LTR of all viral transcripts and features a conserved hairpin structure indispensable for transactivation and viral replication.

### Materials and Methods

### Cell Culture and Reagents

Jurkat (uninfected T-lymphocytes), J1.1 (HIV-1-infected T-lymphocytes), U1 (HIV-1-infected promonocytic) and U937 (promonocytic) cells were cultured in complete RPMI 1640 media with 10% fetal bovine serum (FBS), 1% L-glutamine, and 1% penicillin/streptomycin (Quality Biological, Gaithersburg, MD, USA). Infected cultured central memory CD4 T cells were generated as previously described until day 13 (Macedo, A. B. et al., “Influence of Biological Sex, Age, and HIV Status in an In Vitro Primary Cell Model of HIV Latency Using a CXCR4 Tropic Virus,” AIDS Res. Hum. Retroviruses, 34:769-777 (2018) and was generous gift from Dr. Alberto Bosche (GWU, USA).

A set of primary PBMCs were (Precision For Medicine, Frederick, MD, USA) cultured in vitro first in PMA/GM-CSF for 3 days to obtain monocyte derived macrophages. Cells were then infected with HIV-1 89.6 strain (MOI: 10) for 3 days. Cells were treated with PMA/IL-2. On Day 3 post infection, cells were treated with PMA/IL-7 and a cART cocktail (equal parts of lamivudine (NRTI), tenofovir disoproxil fumarate (NtRTI), emtricitabine (NRTI), and indinavir (protease inhibitor) at 10 μM each). The cART/IL-7 treatment was repeated every other day for the course of one week and cells were harvested for RT-qPCR.

### RNA Isolation and Quantitative Real-Time PCR (RT-qPCR)

For the isolation of total RNA, cells were harvested, washed once in 1×PBS without calcium or magnesium and resuspended in 50 μL of 1×PBS. In this study, total RNA was isolated from the plasma membrane mostly and from Cytoplasm, Nuclear and extracellular vesicles (EVs) in some parts of the study using Trizol Reagent (Invitrogen) as described by the manufacturer's protocol. For RNA estimation, cDNA was generated using GoScript Reverse Transcription Systems (Promega) using Reverse Primers shown in Table 2.

TABLE 2
Reverse Primers.

| SEQ ID | RNA Identity | Sequence for RT Reaction (5′-3′) |
|---|---|---|
| 23 | TAR | CAA CAG ACG GGC ACA CAC TAC (99-119) |
| 24 | TAR1 | CAA CAG ACG GGC ACA CAC TAC (161-184) |
| 25 | TAR2 | CCACTGCTAGAGATTTTCCACACT (161-184) |
| 26 | TAR3 | CCCCCTGGCCTTAACCGAATTTT (387-409) |
| 27 | TAR4 | TATCTCTATCCTTTGATGCACACAA (593-617) |
| 28 | Long LTR | GGGCGCCACTGCTAGAGA (626-643) |
| 29 | Pol | CAAATTTCTACTAATGCTTTTATTTTTTC (2634-2662) |
| 30 | env | TGGGATAAGGGTCTGAAACG (7917-7936) |
| 31 | nef | TGTAAGTCATTGGTCTTAAAGGTACCTGAGG (9010-9040) |

RT-qPCR conditions were as follows: one cycle for 2 min at 95° C. followed by 41 cycles of 95° C. for 15 s and 58° C. for 40 s.

Serial dilutions of DNA from a CEM T-cell line containing a single copy of HIV-1 LAV provirus per cell (8E5 cells) were used as the quantitative standards. The cDNA samples (2 μL per well) for each RNA type were plated into a Master Mix (18 μL per well) containing IQ Supermix (Bio-Rad), TAR Forward Primer (SEQ ID NO: 32:5′-GGT CTC TCT GGT TAG ACC AGA TCT G-3′, 1-25), TAR Reverse Primer (SE ID NO: 33: 5′-CAA CAG ACG GGC ACA CAC TAC-3′ 76-96), and TAR Probe (SEQ ID NO: 34: 5′56-FAM-AG CCT CAA TAA AGC TTG CCT TGA GTG CTT C-36-TAMSp-3′). Reactions were performed in triplicate using the BioRad CFX96 Real-Time System. Quantitation was determined using cycle threshold (Ct) values relative to the 8E5 standard curve using the BioRad CFX Manager Software. All reactions were run in triplicate on the CFX96 Real-Time PCR Detection System (Bio-Rad, Hercules, California 94547, USA) and analysis of generated raw data was analyzed using Microsoft Excel 2016.

### Transfection of HIV-1 DNA

The Cell-Porator™ [Life Technologies, Inc; Bethesda Research Labs (BRL)] was used to transfect the Myeloids (U937) and T-cells (CEM) per the manufacturer's instructions. Briefly, cells (5×10⁶ cells per sample) were electroporated in RPMI media containing 10% FBS and 5% L-glutamine. The cell lines were transfected with pNL4-3 DNA (20 μg) at the following parameters: a capacitance of 800 μF, low resistance, pulse voltage of 230 V and fast charge rate.

### Preparation of Whole Cell Extracts

Jurkat, J1.1, U1, U937 and CEM cells were centrifuged at 1800×RPM at room temperature for 5 min. Cell pellets were washed with 1× Phosphate Buffer Saline (PBS) without Ca++ and Mg++ and resuspended in lysis buffer comprising 120 mM NaCl, 50 mM Tris-HCl, pH 7.5, 50 mM NaF, 5 mM EDTA, 0.5% NP-40 (NP40), 0.2 mM Na₃VO₄, 1 mM DTT and 1 complete protease inhibitor cocktail tablet/50 mL (Roche Applied Science, Mannheim, Germany). Cell pellets were incubated on ice for 20 min with vortexing every 5 min. The whole cell lysate was separated from cell debris by centrifugation at 10,000×RPM at 4° C. for 10 min. Bradford assay (Bio-Rad) was used to determine the protein concentration according to the manufacturer's protocol.

### Western Blot Analysis and Antibodies

Laemmli buffer (Tris Glycine SDS and β-mercaptoethanol) was added to whole cell lysate samples. Samples were heated at 95° C. for 3 min, then loaded into 4-20% Tris-glycine gels (Invitrogen) with a Precision Plus Protein™ Standard (BioRad) and fractionated at 150 V. Gels were transferred onto Immobilon Polyvinylidene fluoride (PVDF) membranes (Millipore) at 50 milliamps overnight. Five percent milk in PBS containing 0.1% Tween-20 (PBS-T) was used to block the protein membranes for 2 h at 4° C. Primary antibody in PBS-T; HSP90 (Novus Biologicals, NB100-1972) was added to the membranes prior to overnight incubation at 4° C.; primary antibodies include α-p24 were obtained from NIH AIDS Reagent Program, Manassas, VA, USA. Membranes were washed three times with PBS-T (5 min/wash). Complementary HRP-conjugated secondary antibodies were added, followed by incubation for 2 h at 4° C. Membranes were washed twice with PBS-T and once with PBS, 5 min per wash. HRP luminescence was activated with Clarity Western ECL Substrate (Bio-Rad). Membranes were developed using the Molecular Imager ChemiDoc Touch system (Bio-Rad).

### Plasma Membrane Extraction

Cells were cultured in growth media and spun down at 600×g for 5 min at 4° C. Cell pellets were washed with ice-cold 1× Phosphate Buffer Saline (PBS) without Ca++ and Mg++. Plasma membranes were isolated using the Plasma Membrane Protein Extraction Kit (ab65400, Abcam) from Abcam. Briefly, Cell pellets were resuspended in 2 mL of Homogenize Buffer (HB) Mix. Cell suspension was Dounce Homogenized on ice for 50-60 strokes and then, spun at 700×g for 10 min at 4° C. This pellet was the nuclear fraction and supernatants were transferred to new tubes and spun again at 10,000×g for 30 min at 4° C. The pellets generated from this spin were crude membranes and the supernatant was soluble cytosol.

### Immunoprecipitation

Five micrograms of each IgG and J2 (RNA binding antibody; Scicons; Cat #J2) were used for immunoprecipitation. In another reaction, 1 μg of Human rec-hSiglec-14 (R&D Systems; cat #4905-SL-050) was added to the samples (overnight) prior to immunoprecipitation with Siglec-5/Siglec-14 antibody (5 ug, Biorad; cat #MCA5787GA). Protein A/G beads were added the next day (2 hours, at 4° C.) and the IPed samples were washed. RNA was isolated for RT-qPCR using TAR primers. An IP with IgG antibody served as a control.

### Sialidase Enzymatic Digestion

To digest the Glycans, 0.8-1 ug of RNA was incubated with 1 ul of Sialidase enzyme (New England Biolabs (NEB) Cat #P0722) in GlycoBuffer 1 (10×) and reaction was incubated at 37° C. for 1 hour. After one hour, enzyme was heat deactivated by keeping the sample at 65° C. for 10 minutes and processed samples were used for downstream RT/qPCR reactions.

### Flow Cytometry

1×10⁶ cells from each cell type were centrifuged, washed and fixed with 70% alcohol and Anti-Fc antibody dilution was used to prevent nonspecific binding of primary antibody, stained using a primary antibody against dsRNA (anti-dsRNA J2) followed by a donkey anti-mouse Alexa-488 secondary antibody and analyzed using a BD FACSaria II Flow Cytometer.

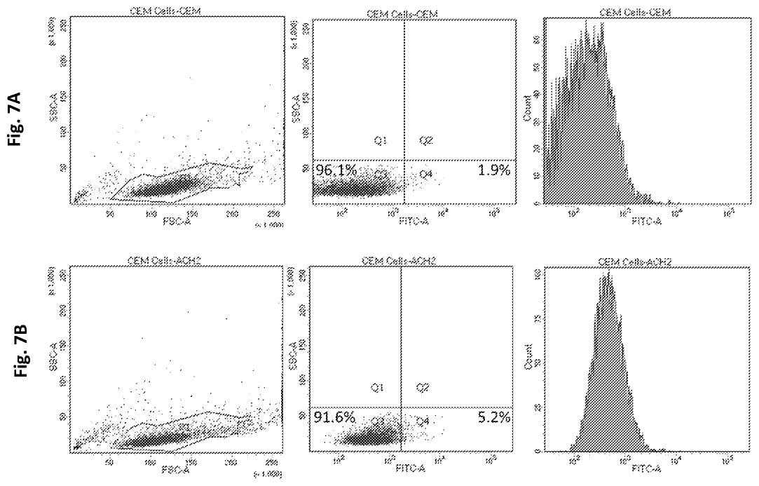

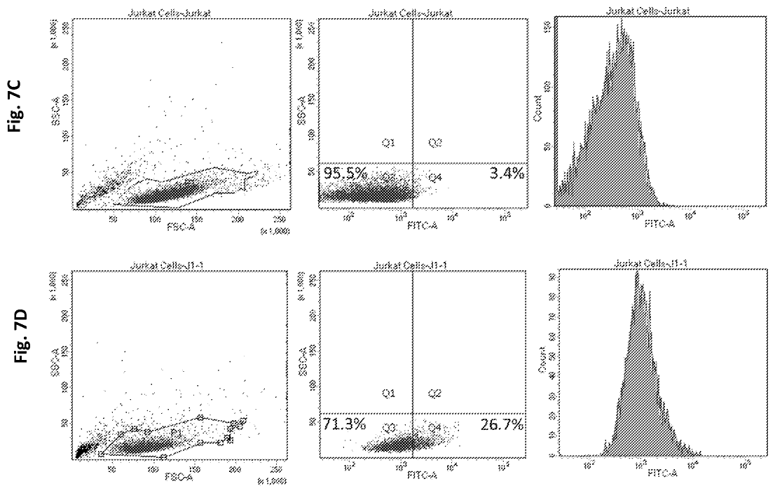

Total dsRNA expression level was compared between uninfected and infected T-cells using Flow Cytometry. Briefly, CEM and Jurkat cells were used to determine expression levels of dsRNA on intact human T-cells following HIV infection using Anti-dsRNA monoclonal antibody J2. Samples were stained using a primary antibody against dsRNA (anti-dsRNA J2) followed by a donkey anti-mouse Alexa-488 secondary antibody and analyzed using a BD FACSaria II Flow Cytometry. Data in FIGS. 7A-7B compares uninfected CEM cells (Panel A) to infected ACH2 cells (Panel B), where there was an increase in dsRNA population by 3.3% on the surface of the infected cells. We then tested another set of uninfected (Jurkat) to infected (J1.1) cells, and observed a larger increase by 23.3% in infected cells (Panels C and D). Collectively, this data indicated potential presence of increased dsRNA populations on the surface of infected cells.

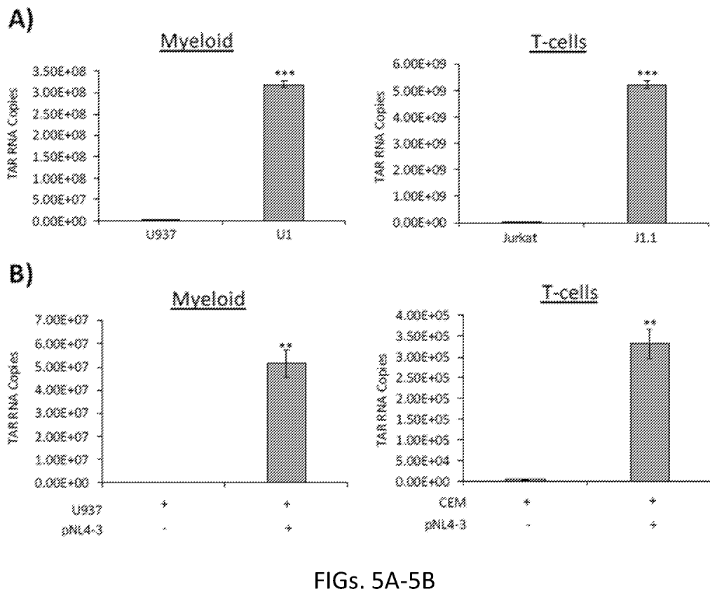

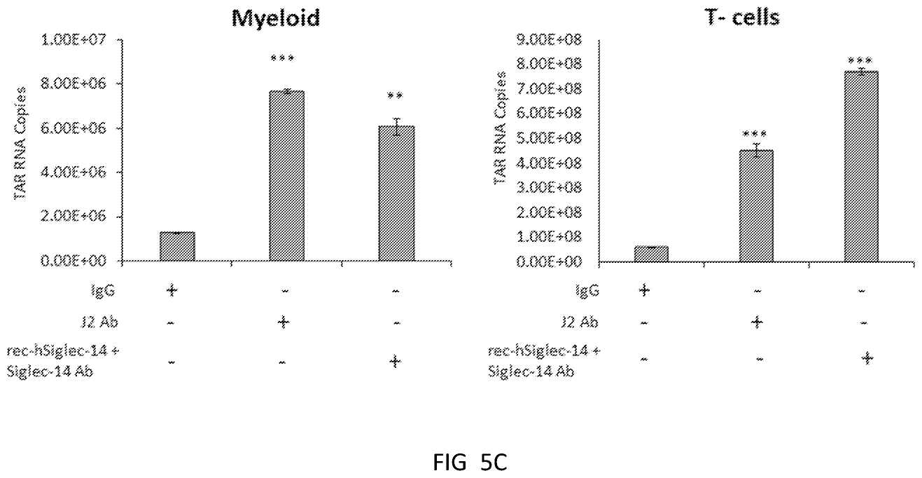

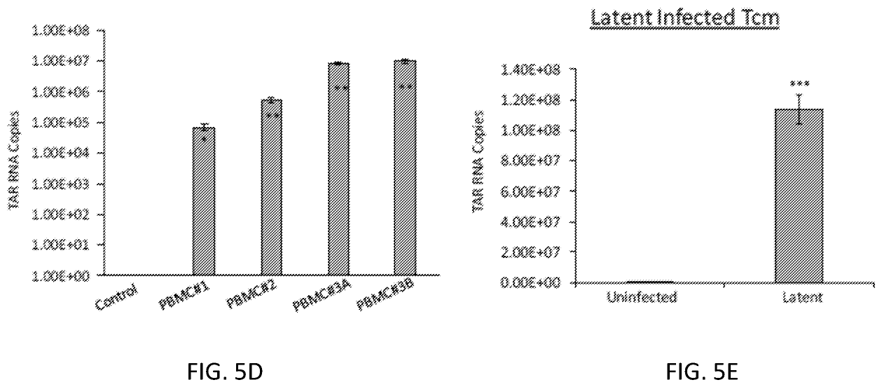

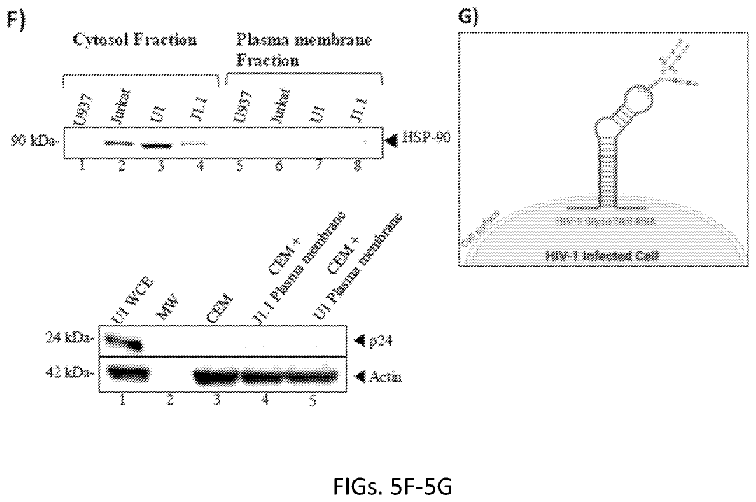

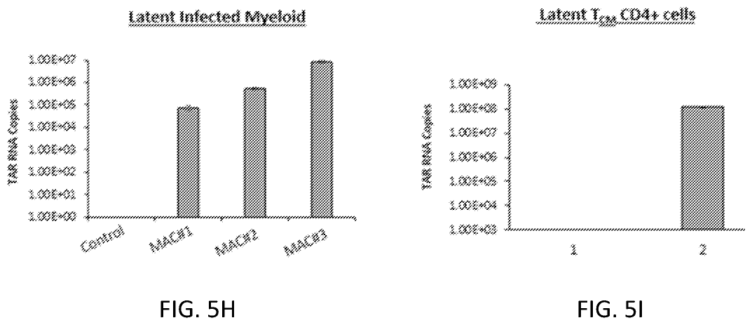

To detect TAR RNA on the membrane, U1 and J1.1 cells (both infected) were used for membrane isolation, followed by PCR. Plasma membranes were isolated from ˜50 million cells of monocytes (U937), uninfected T-cells (Jurkat), HIV-1 infected monocytes (U1) and T-cells (J1.1) following the plasma membrane Extraction Kit protocol (abcam; 65400). RNA was isolated from plasma membrane pellets using Trizol Reagent, deglycosylated followed by RT-qPCR to quantify the TAR RNA copies in plasma membrane. Student's t-tests used to calculate the statistical significance between uninfected and infected cells. *, p<0.05; **, p<0.01; ***, p<0.001. Error bars, S.D. Data in FIG. 5A shows significant amount of TAR in these fractions.

pNL4-3 DNA was then transfected into U937 and CEM cells and membrane fractions were isolated 48 hrs later. Myeloid cells (U937) and T-cells (CEM) were infected with HIV-1pNL4-3 at 37° C. for 48 hours at MOI of 0.1 followed by plasma membrane isolation, deglycosylated and RT-qPCR to confirm the translocation of TAR RNA in the plasma membrane of transfected cells. Data in FIG. 5B show the presence of TAR RNA in the membrane fractions from the transfected cells.

Next, both infected U1 and J1.1 membranes were used for immunoprecipitation using cither RNA antibody (J2), glycosylated RNA antibody (siglec-5/siglec-14) or Control IgG. To test for the presence of Siglec (Glycans) molecules on TAR RNA surface, plasma membranes were isolated from infected Myeloids (U1) and T-cells (J1.1) and prepared for immunoprecipitation (IP). Each preparation was fractioned into three reactions. Five micrograms of each IgG and J2 (RNA binding antibody; Scicons) were used for IP in the first two reactions. In the third reaction, 1 ug of Human rec-hSiglec-14 was added to the samples (overnight) prior to immunoprecipitation with Siglec-5/Siglec-14 antibody (5 ug). Protein A/G beads were added the next day (2 hours, at 4° C.) and the IPed samples were washed. RNA was isolated for RT-qPCR using TAR primers. Data in FIG. 5C show the presence of both RNA and glycosylated RNA in the membrane.

PBMC cells were then infected and spreading of infection was allowed, followed by CART treatment. Adherent cells (mostly myeloid) were processed for membrane purification and presences of TAR. To examine the presence of TAR RNA in the plasma membrane of latent PBMCs from three different donors (sample 3 was divided into 2 samples due to high cell numbers) were infected with HIV-1 (89.6) and 3 days later treated with cART and IL-7 for one week.

To test for presence of RNA in the plasma membrane fractions isolated from primary latent T_{CM} CD4+ cells, 5×10⁶ naive CD4+ T cells from HIV+donors were isolated by magnetic negative selection (Stemcell Technologies) and cultured at 10⁶ cells/ml in 96-well plates using RPMI 1640 medium supplemented with 10% fetal bovine serum, 2 mM l-glutamine, 100 units/ml penicillin, and 100 g/ml streptomycin supplemented with 12.5 ul/ml of Dynabeads human T-activator CD3/CD28) (Invitrogen), 2 μg/ml anti-human IL-12 (PeproTech), 1 μg/ml anti-human IL-4 (PeproTech), and 10 ng/ml transforming growth factor β1 (TGF-β1). After 3 days, Dynabeads were removed by magnetic selection, and cells were washed, followed by culture in R-10 medium supplemented with 30 IU/ml of IL-2 (R-10-30). The medium was changed on days 4 and 5 with fresh R-10-30 medium.

Adherent cells were collected 24 hrs after PMA treatment. Plasma membranes were isolated from PBMCs and subjected to total RNA isolation, followed by RT-qPCR using TAR primers. Student's t-tests was done to calculate the statistical significance w.r.t control ***, p<0.001. Error bars, S.D. The results in FIG. 5E show a significant amount of TAR RNA on the membrane as compared to negative control (see also FIG. 5I). Data in FIG. 5D show presence of TAR from PBMC donors; see also FIG. 5J (three PBMC donors (all infected macrophages showed significant TAR RNA copies˜10⁵ to 10⁷ in the plasma membrane under latent phenotype)). These results indicate the presence of TAR RNA in plasma membrane of infected T-cells and macrophages.

Membrane fractions of latent Tem cells were isolated for presence of TAR RNA (FIGS. 5E and 5I). To test for purity of the membrane fractions, western blots were performed for lack of HSP-90 (FIG. 5F; Top image) and co-culture of the membrane fractions for presence of potential viral particle contamination, where no infection of CEM recipient cells was observed (FIG. 5F; Bottom image). Briefly, purified latent TCM CD4+ cells (5 million) using the Dynabeads CD4 Positive Isolation Kit (Thermo Fisher Scientific) were used for plasma membrane isolation, RNA isolation and RT-qPCR using TAR primers. Student's t-tests was done to calculate the statistical significance between latent infected and uninfected T-cells. *, p<0.05; **, p<0.01; ***, p<0.001. FIG. 5F; Top image: Plasma membrane and cytosolic fractions (25 ug) from uninfected Myeloid/T-cells (U937, Jurkat) and infected Myeloid/T-cells (U1, J1.1) were immunoblotted for the cytosolic marker, HSP-90 protein (NB100-1972). Absence of HSP-90 proteins bands in plasma membrane fractions confirms a clear separation of plasma membrane from cytosolic fraction. FIG. 5F; Bottom image: Examination of the potential infectious nature of the plasma membrane isolations from infected cells. Uninfected CEM cells were treated with plasma membrane fractions derived from J1.1 or U1 cells (25 ul=˜109 copies of TAR) and left in culture for 72 hrs. Total cell lysate were then immunoblotted for p24. U1 extract served as control.

Diagram of potential glycosylated TAR (G-residues) on infected cells is schematically depicted in FIG. 5G. Collectively, these data show that TAR RNA is present on the surface of cells (FIG. 5G) and may be used to target infected cells. Here, plasma membrane fractions were isolated from J1.1 (HIV-1 infected T-cells) and U1 (HIV-1 infected Myeloid) cells and examined for the presence of HIV-1 TAR RNA via RT/qPCR. The plasma membrane preparations were subjected to sialidase enzymatic digestion (see method section) prior to RT reaction to remove potential sugar moieties, if any, attached to RNA molecules that could hinder the RNA conversion to cDNA. The plasma membrane fractions from the parent uninfected cells (Jurkat and U937) were utilized as controls. The results demonstrated the presence of 3.19×10⁸ and 5.24×10⁹ copies of TAR RNA in myeloid and T cells, respectively (FIG. 5A). Furthermore, to confirm that RNA was not being isolated from Cytosol, the purity of the plasma membrane and cytoplasmic fractions was examined using immunoblotting for HSP-90. HSP-90 is a cytosolic marker and the absence of this protein in FIG. 5F, Top image, indicates the relative purity of the isolation method for plasma membrane. In conclusion, the RT/qPCR data demonstrated the presence of HIV-1 TAR RNA in the plasma membrane of the infected T-cells and Myeloids.

The data in FIG. 5A was performed with HIV-1 infected cell lines containing integrated HIV-1 proviral DNA (J1.1 and U1). Next, it was determined whether viral RNA can also be found in the plasma membrane of the HIV-1 transfected cells. To measure the TAR RNA copies in the plasma membrane of the transfected cells, uninfected Myeloid (U937) and T-cells (CEM) were transfected with HIV-1_{pNL4-3} DNA (20 ug) for 48 hours at 37° C. Then the plasma membrane fractions were isolated, the fractions were deglycosylated, and RT-qPCR was performed to confirm the translocation of TAR RNA in the plasma membrane of the transfected cells. Results in FIG. 5B indicated that plasma membrane fractions from the transfected Myeloids and T-cells contain 5.13×10⁷ and 3.31×10⁵ copies of TAR RNA respectively. Next, to examine the potential infectious nature of the plasma membrane isolations from infected cells, uninfected CEM cells were treated with plasma membrane fractions derived from J1.1 or U1 cells and incubated for 72 hrs. Total cell lysate was then immunoblotted for the presence of p24 (viral protein). U1 extract (FIG. 5F, Lower image, Lane 1, Top insert) served as positive control. As shown in FIG. 5F, Lower image, CEM cells treated with plasma membrane fractions from J1.1 (Lane 4) or U1 cells (Lane 5) were negative for presence of newly replicating p24 viral antigen indicating that these fractions were not infectious in nature. Collectively, transfection data showed the presence of TAR RNA in the plasma membrane of cells. Furthermore, plasma membrane fractions derived from HIV-1 infected cells were negative for the presence of infectious virions.

To determine whether the TAR RNA is glycosylated and present in the plasma membrane, plasma membrane from Myeloids and T-cells were isolated, and each preparation was fractioned into three independent reactions. Five micrograms of each IgG and J2 antibody were used for Immunoprecipitation in the first two reactions. J2 anti-double-stranded RNA (dsRNA) antibody binds specifically to double strand regions of RNA and often used to identify cells infected with RNA viruses. In the third reaction, 1 μg of Human rec-hSiglec-14 was added to the samples (overnight) prior to immunoprecipitation with Siglec-5/Siglec-14 antibody (5 ug). Siglec-14 is a glycan-recognition protein that is expressed on myeloid cells. Protein A/G beads were added the next day (2 hours, at 4° C.) and the IPed samples were washed. RNA was isolated for RT-qPCR using TAR primers. Use of the J2 antibody represents the total TAR RNA transcripts in the plasma membrane and Siglec-14 antibody represents the Glycosylated TAR RNA. The results in FIG. 5C demonstrated that, in myeloid cells, 74.7% of total TAR RNA on the cell surface is glycosylated TAR RNA. In contrast, in T-cells, 100% TAR RNA transcript were glycosylated (FIG. 5C, Lane 2) since glycosylated TAR RNA transcripts were greater than total RNA transcripts (FIG. 5C, Lane 3). Taken Together, TAR RNA contain Siglecs molecules and present in the plasma membrane of infected cells.

### Example 3

### HIV-1 Promoter Makes 4 Distinct Non-Coding RNAs

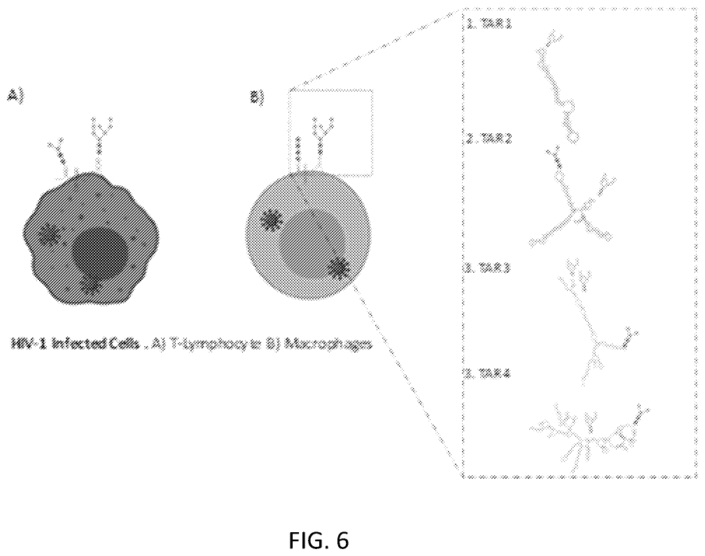

Other than the normal genomics RNA, HIV-1 promoter makes 4 other distinct RNA molecules from the 5′ LTR, all of which are non-coding RNAs. Sequencing results identified 4 main clusters of sequences ending at ˜96, 184, 408, and 615 bp from the transcription start site (+1; FIG. 6).

RNA sequence analysis was used to define the 3′ ends of these 4 transcripts, which were the result of paused RNA Polymerase II (Pol II) on the HIV-1 genome. The first 2 clusters were found to end within the LTR region, whereas the last 2 clusters were found to end in the Gag region. Using transfer of the TAR-gag RNA into recipient cells, we observed no new synthesis of HIV-1 p24/p17 proteins, indicating that TAR-gag are long non-coding RNAs.

One or all 4 of these RNAs may be on the membrane. TAR1 seems to be the most predominant RNA using infected cell lines and purified patient samples.

The experimental data above shows viral RNAs transcribed from the 5′ LTR on the plasma membrane in HIV-1 infected cells. Additionally, HIV-1 has been shown to produce antisense transcripts from 3′ LTR, which promotes viral latency. We also propose the presence of these antisense transcripts transcribed from the 3′ LTR region on the cell surface such as antisense transcripts ASP RNA in HIV-1 and HBZ RNA in HTLV-1.

### Example 4

### Viral RNA Distribution in HIV-1 Infected Cells

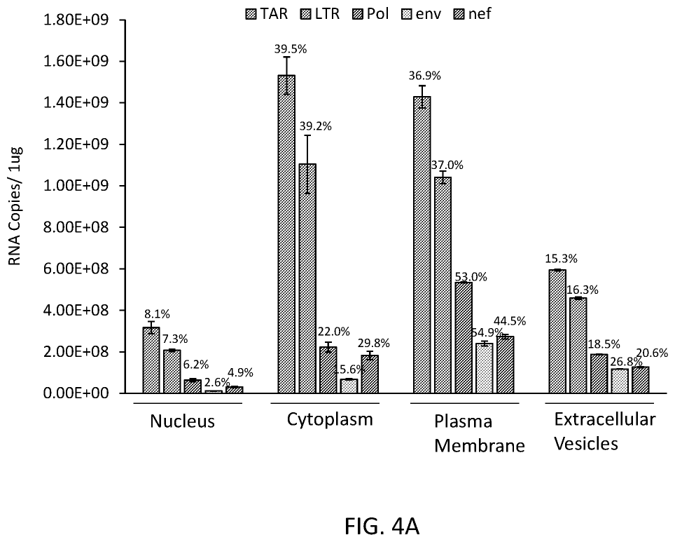

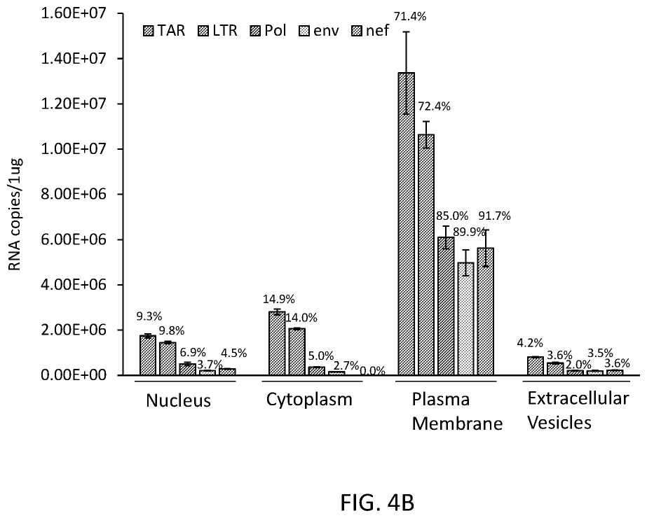

To determine the type of RNAs distributed across various cell compartments, RNA was isolated from 4 different compartments of the cell; Nucleus, Cytoplasm, Plasma membrane and Extracellular vesicles from infected T-lymphocytes and Myeloid cells. Total RNA was analyzed by RT-qPCR for the presence of TAR, Long LTR, Pol, env and nef transcripts using primers from Yukl et al., “HIV Latency in Isolated Patient CD4+ T Cells May Be Due to Blocks in HIV Transcriptional Elongation, Completion, and Splicing,” Sci. Transl. Med., 2018, 10, eaap9927 (2018) in all four aforementioned cell compartments. During HIV-1 transcription, all HIV transcripts contain the TAR region, and the quantity of TAR-containing transcripts over total transcripts indicates transcription Initiation. Long LTR represents aborted elongation beyond 5′ LTR. Pol represents unspliced HIV-1 transcripts, env represents doubly spliced transcripts and nef represent distal transcription. In myeloid cells (FIG. 4B) all the transcripts were enriched in plasma membrane fractions, followed by cytoplasm, nucleus, and extracellular vesicles fractions. In contrast, in T-cells (FIG. 4A), TAR and long LTR transcripts were abundant in cytoplasmic fractions, other transcripts including Pol, env and nef were highly enriched in Plasma membrane fractions similar to Myeloid cells. Unlike Myeloid cells, extracellular vesicle fractions from T-cells contains high copy number of transcripts as compared to nuclear fractions. The differential transcription behavior between Myeloid and T-cells is in agreement with previously published data. However, overall plasma membrane fractions enriched with viral transcripts points to potential assembly of virions or simple incorporation of RNA in the plasma membrane.

### Example 5

### Presence of Various TAR Non-Coding RNA in Plasma Membrane of HIV-1 Infected Cells

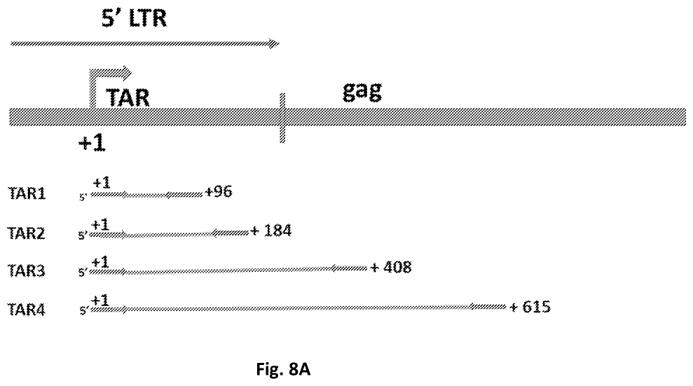

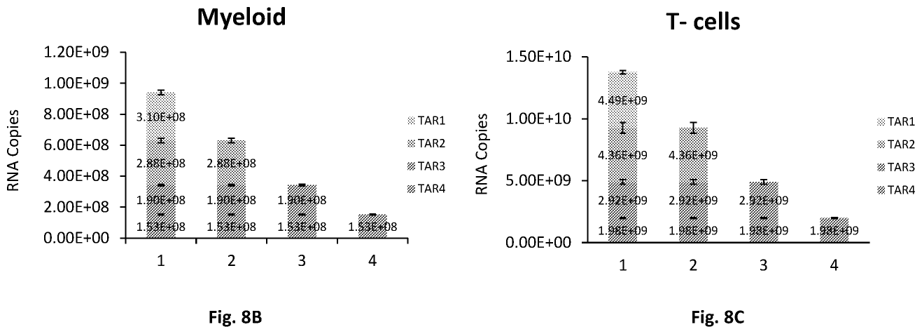

Other than the normal genomics RNA, HIV-1 promoter makes four small distinct RNA molecules from the 5′ LTR, all of which are non-coding RNAs and byproducts of aborted transcription at different intervals and not degradation products of full-length viral transcripts. In this study RNA sequence analysis was used to define the 3′ ends of these 4 short TAR transcripts. The length and location of the four transcripts named as TAR1, TAR2, TAR3 and TAR4 are shown in FIG. 8A. Next, the presence of four distinct non-coding RNA transcripts in the plasma membrane of the HIV-1 infected Myeloid and T-cells were determined. To do so, RNA was isolated from the plasma membrane preparation of both cell types (FIGS. 8B-8C) and analyzed for 4 short TAR transcripts using RT/qPCR and specific primers described in method section. Interestingly, both cell types contain all four non-coding RNA transcripts in the plasma membrane fraction with TAR1 being abundant followed by TAR2, TAR3 and TAR4. Additionally, T-cells contained mostly high copy number of non-coding transcripts as compared to infected Myeloids. The RNA copies for each transcript is shown in Table 3.

TABLE 3
Levels of various non-coding TAR RNAs in HIV-1 infected cells.

RNA Copies

| Non-coding RNA Identity | Myelod Cells | T- cells |
|---|---|---|
| TAR1 | 3.10 × 10⁸ | 4.49 × 10⁹ |
| TAR2 | 2.88 × 10⁸ | 4.36 × 10⁹ |
| TAR3 | 1.90 × 10⁸ | 2.92 × 10⁹ |
| TAR4 | 1.53 × 10⁸ | 1.98 × 10⁹ |

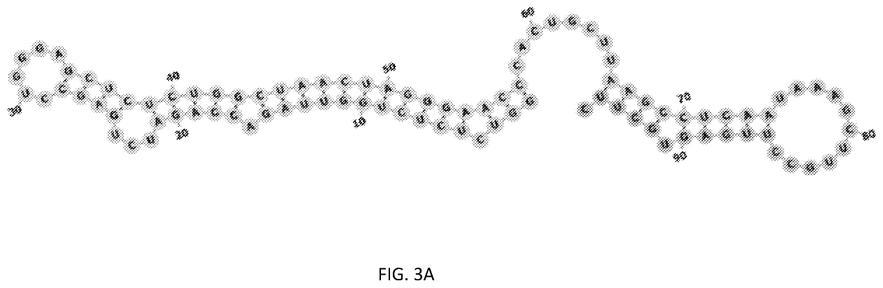

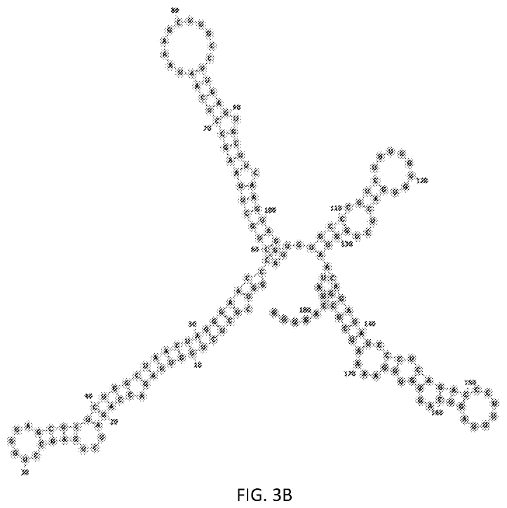

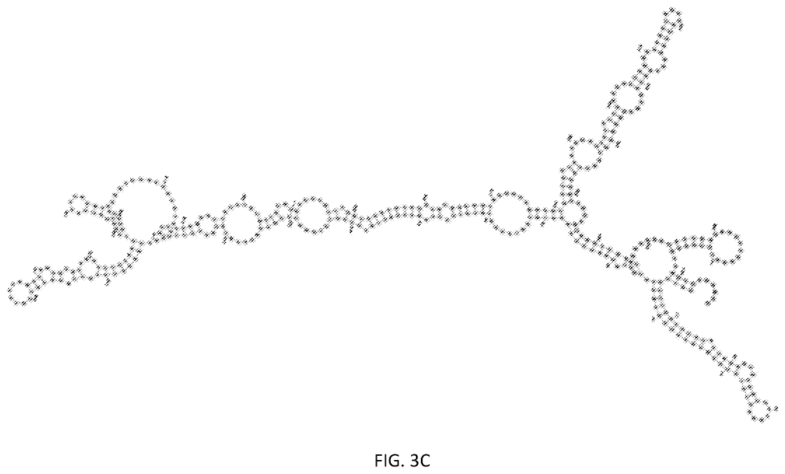

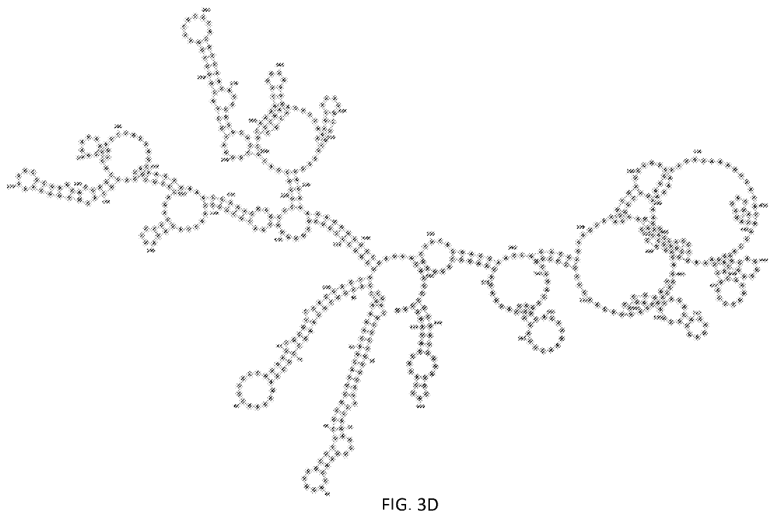

The secondary structure of all four non-coding transcripts are shown in FIG. 3A-3D (TAR1≥TAR2>TAR3>TAR4). Therefore, plasma membrane of both myeloid and T-cells contain non-coding viral transcripts of different length.

### Enumerated Embodiments

The following enumerated embodiments are provided, the numbering of which is not to be construed as designating levels of importance.

Embodiment 1 provides a composition comprising an extracellular portion, or a molecule that specifically binds the extracellular portion, of a viral nucleic acid present on a plasma membrane of a host cell infected with a virus.

Embodiment 2 provides the composition of embodiment 1, wherein the viral nucleic acid is expressed in a latent life cycle of the virus.

Embodiment 3 provides the composition of embodiment 1 or 2, wherein the viral nucleic acid comprises a noncoding RNA.

Embodiment 4 provides the composition of any one of embodiments 1-3, wherein the viral nucleic acid is glycosylated.

Embodiment 5 provides the composition of any one of embodiments 1-4, wherein the virus is a retrovirus.

Embodiment 6 provides the composition of any one of embodiments 1-5, wherein the virus is a human immunodeficiency virus (HIV).

Embodiment 7 provides the composition of any one of embodiments 1-6, wherein the virus is a HIV-1.

Embodiment 8 provides the composition of any one of embodiments 1-7, wherein the viral nucleic acid comprises a trans-activation response element (TAR).

Embodiment 9 provides the composition of any one of embodiments 1-7, wherein the viral nucleic acid comprises the nucleotide sequence as set forth in any one of SEQ ID NOS: 1-22; or a variant sequence thereof.

Embodiment 10 provides the composition of any one of embodiments 1-9 further comprising a pharmaceutically acceptable carrier and/or adjuvant.

Embodiment 11 provides the composition of any one of embodiments 1-10, wherein the molecule comprises an antibody or aptamer.

Embodiment 12 provides the composition of any one of embodiments 1-11, wherein the molecule comprises a diagnostic or therapeutic moiety.

Embodiment 13 provides the composition of any one of embodiments 1-12, wherein the composition is an exosome, liposome, macrovesicle, or nanoparticle.

Embodiment 14 provides the composition of any one of embodiments 1-13, wherein the composition further comprises one or more elements of a gene editing system.

Embodiment 15 provides a method for treating or preventing infection by a virus, the method comprising administering a therapeutically or prophylactically effective amount of the composition of any one of embodiments 1-14 to a subject, thereby treating or preventing the infection by the virus in the subject.

Embodiment 16 provides the method of embodiment 15, wherein the composition is administered to the subject during a latent life cycle of the virus.

Embodiment 17 provides the method of embodiment 15 or 16, wherein the subject has tested positive for HIV infection but has no symptoms associated with HIV.

Embodiment 18 provides the method of embodiment 15 or 16, wherein the subject has acquired immunodeficiency syndrome (AIDS).

Embodiment 19 provides a method for identifying a subject infected with a virus, the method comprising detecting the presence of an extracellular portion of a viral nucleic acid present on a plasma membrane of a host cell infected with a virus using the composition of any one of embodiments 1-14, thereby identifying the subject infected with the virus.

Embodiment 20 provides the method of embodiment 19, wherein the detecting step is during a latent life cycle of the virus.

Embodiment 21 provides the method of embodiment 19 or 20, wherein the host cell infected with the virus is from a subject having tested positive for HIV infection but has no symptoms associated with HIV.

Embodiment 22 provides the method of embodiment 19 or 20, wherein the host cell infected with the virus is from a subject having acquired immunodeficiency syndrome (AIDS).

Embodiment 23 provides a method for separating or killing virally infected cells ex vivo, the method comprising contacting cells obtained from a subject with the composition of any one of embodiments 1-14, wherein the virally infected cells bind to the molecule.

Embodiment 24 provides the method of embodiment 23, wherein the contacting step comprises fluorescence-activated cell sorting (FACS).

Embodiment 25 provides the method of embodiment 23 or 24, wherein the cells are obtained from the subject during a latent life cycle of the virus.

Embodiment 26 provides the method of any one of embodiments 23-25, wherein the subject has tested positive for HIV infection but has no symptoms associated with HIV.

Embodiment 27 provides the method of any one of embodiments 23-25, wherein the subject has acquired immunodeficiency syndrome (AIDS).

Embodiment 28 provides the method of any one of embodiments 23-27, where the infected cells are in a transfusion.

Embodiment 29 provides the method of any one of embodiments 23-27, the infected cells are in a bone marrow or solid organ transplant.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising an extracellular portion, or a molecule that specifically binds the extracellular portion, of a viral nucleic acid present on a plasma membrane of a host cell infected with a virus.

2. The composition of claim 1, wherein the viral nucleic acid is expressed in a latent life cycle of the virus.

3. The composition of claim 1, wherein the viral nucleic acid comprises a noncoding RNA.

4. The composition of claim 1, wherein the viral nucleic acid is glycosylated.

5. The composition of claim 1, wherein the virus is a retrovirus.

6. The composition of claim 1, wherein the virus is a human immunodeficiency virus (HIV).

7. The composition of claim 1, wherein the virus is a HIV-1.

8. The composition of claim 1, wherein the viral nucleic acid comprises a trans-activation response element (TAR).

9. The composition of claim 1, wherein the viral nucleic acid comprises the nucleotide sequence as set forth in any one of SEQ ID NOs: 1-22; or a variant sequence thereof.

10. The composition of claim 1 further comprising a pharmaceutically acceptable carrier and/or adjuvant.

11. The composition of claim 1, wherein the molecule comprises an antibody or aptamer.

12. The composition of claim 1, wherein the molecule comprises a diagnostic or therapeutic moiety.

13. The composition of claim 1, wherein the composition is an exosome, liposome, macrovesicle, or nanoparticle.

14. The composition of claim 1, wherein the composition further comprises one or more elements of a gene editing system.

15. A method for treating or preventing infection by a virus, the method comprising
administering a therapeutically or prophylactically effective amount of the composition of claim 1 to a subject, thereby treating or preventing the infection by the virus in the subject.

16. The method of claim 15, wherein the composition is administered to the subject during a latent life cycle of the virus.

17. The method of claim 15, wherein the subject has tested positive for HIV infection but has no symptoms associated with HIV.

18. The method of claim 15, wherein the subject has acquired immunodeficiency syndrome (AIDS).

19. A method for identifying a subject infected with a virus, the method comprising
detecting the presence of an extracellular portion of a viral nucleic acid present on a plasma membrane of a host cell infected with a virus using the composition of claim 1, thereby identifying the subject infected with the virus.

20-22. (canceled)

23. A method for separating or killing virally infected cells ex vivo, the method comprising
contacting cells obtained from a subject with the composition of claim 1, wherein the virally infected cells bind to the molecule.

24-29. (canceled)
